(19) United States
(12) Patent Application Publication
(10) Pub. No.: US 2017/0197935 A1
(43) Pub. Date: Jul. 13, 2017

(54) INHIBITORS OF DNA GYRASE FOR THE TREATMENT OF BACTERIAL INFECTIONS

(71) Applicant: Vitas Pharma Research Private Limited, Gachibowli (IN)

(72) Inventors: Chandrasekhar Alapati, Hyderabad (IN); Surobhi Lahiri, Hyderabad (IN); Radha Rangarajan, Hyderabad (IN); Rajinder Kumar, Cambridge (GB)

(21) Appl. No.: 15/467,820

(22) Filed: Mar. 23, 2017

Related U.S. Application Data
(63) Continuation-in-part of application No. 14/434,870, filed on Apr. 10, 2015, now Pat. No. 9,643,979.

(30) Foreign Application Priority Data
Oct. 10, 2012 (IN) 2795/CHE/2012

(51) Int. Cl. C07D 401/06; C07D 471/04

(52) U.S. Cl. CPC C07D 401/06; C07D 471/04

(57) ABSTRACT

The present invention relates to compounds which specifically inhibit bacterial DNA Gyrase and can be used for the treatment of respiratory tract infections.

### CROSS-REFERENCE TO RELATED APPLICATIONS

The present disclosure is a continuation-in-part of US application National Phase application Ser. No. 14/434,870, filed, 10 Apr. 2015, which is entitled to the benefit under 35 U.S.C. §120 and 365(c) of International Patent Application PCT/IB2013/059192, entitled, “Inhibitors of DNA Gyrase for the treatment of bacterial infections”, filed, 8 Oct. 2013, which claims priority to Indian Patent Application 2795/CHE/2012, filed, 10 Oct. 2012, all of which are incorporated herein by reference in their entirety.

### FIELD OF THE INVENTION

This invention is related to compounds which specifically inhibit bacterial DNA Gyrase for the treatment of bacterial infections.

### BACKGROUND OF THE INVENTION

Antibacterial drug resistance is a worldwide problem; new mechanisms of resistance emerge periodically and spread rapidly across the globe. The growing rate of antimicrobial resistance in clinical and non clinical settings poses significant threat to human health and animals, not only in India but also globally (Lancet Infectious Diseases, 9, 228-36, 2009). Each mechanism of resistance renders yet another class of antibiotics ineffective, ultimately resulting in fewer and fewer therapeutic options for patients. In fact, WHO now recognizes antimicrobial resistance as one of three greatest threats to human health (Clinical Infectious Diseases 50, 1081-1083, 2010). To address the issue of drug resistance, new chemotypes that target critical pathways in bacteria must be developed. We have identified a novel series of compounds that inhibit DNA Gyrase, a member of the DNA Topoisomerase family, and have broad spectrum antimicrobial activity.

DNA Topioisomerases are involved in the transient breaking and rejoining of DNA during replication, transcription and recombination. They are well conserved across the bacterial species and essential for viability. There are two classes of Topoisomerases, depending on whether they introduce single stranded (type 1) or double stranded breaks (type 2). DNA Gyrase and Topo IV are Type 2 Topoisomerases. Gyrase is responsible for the introduction of negative supercoils into DNA to allow fork progression during replication. It is a heterodimer consisting of two subunits of GyrA and two subunits of GyrB (Reviewed in Infectious Disorders—Drug Targets 7, 3-9, 2007).

Gyrase is a clinically validated target. Inhibitors of this target, the fluoroquinolones have been in use since the 1960s but suffer widespread drug resistance. Despite extensive research, newer generations of fluorquinolones have not overcome resistance effectively. Recently two non-fluoroquinolone inhibitors of Gyrase have been described. One of them is NXL101 and the other is GSK299423. NXL101 belongs to a novel quinoline class with potent activity against gram-positive bacteria, including methicillin- and fluoroquinolone-resistant strains (Antimicrobial Agents and Chemotherapy, 52, 3339-3349, 2008). GSK299423 shows potent antibacterial activity against MRSA, fluoroquinolone resistant strains of S. aureus and Gram negatives such as E. coli, H. influenzae, M. catarrhalis and Klebsiella pneumoniae (Nature, 466, 935-942, 2010). While the compound potently inhibits DNA Gyrase, it has serious hERG binding liability (BMCL, 21, 7489-7495, 2011). Similarly, NXL-101 causes QT prolongation, which led to its discontinuation from clinical development (North American Journal of Medical Science, 4, 537-47, 2012). Nevertheless, the target continues to be attractive and novel chemotypes directed against get will have significant clinical benefits, once proven to be efficacious and safe.

### BRIEF DESCRIPTION OF DRAWINGS

FIG. 1 discloses time kill kinetics of VT-03-00061 against MRSA 33591.

FIG. 2 discloses time kill kinetics of VT-03-00061 against E. coli 25922.

### DETAILED DESCRIPTION OF THE INVENTION

The present invention provides compounds of formula (I) or pharmaceutically acceptable salts thereof:

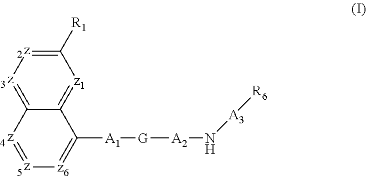

wherein,
Z₁, Z₂, Z₃ are each independently CR₁;
Z₄, Z₅, Z₆ are each independently selected from a group consisting of N or CR₁;
Z₂ and Z₃ together form an optionally substituted saturated or unsaturated 5-6 membered cyclic ring which contains minimum one heteroatom at bridging or any other position of the ring;
Z₅ and Z₆ together form an optionally substituted saturated or unsaturated 5-6 membered cyclic ring which contains minimum one heteroatom at bridging or any other position of the ring;
Z₄ and Z₆ directly form a bond in absence of Z₅ where its substitution together form an optionally substituted saturated or unsaturated 5-6 membered cyclic ring containing at least one heteroatom at bridging or any other position of the ring;
R₁ are each independently selected from a group consisting of hydrogen, oxo, cyano, halogen, hydroxyl and C₁₋₆ alkyl optionally substituted with one or two C₁₋₆ alkoxy;
A₁ is selected from a group consisting of —(CR₂R₃)ₘ—CH₂—, —CH₂—(CR₂R₃)ₘ—, —NH—(CR₂R₃)ₘ—CH₂, —(CR₂R₃)ₘ—CH₂—O— and

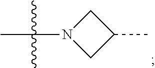

wherein, m is for 2;
————— is connectivity to G;
R₂ is selected from a group consisting of hydrogen, halogen, hydroxyl, acyloxy, C₁₋₆ alkyl optionally substituted with one or two C₁₋₆ alkoxy and C₁₋₆ alkoxy optionally substituted with C₁₋₆ alkyl
R₃ is hydrogen;
G is selected from a group of formulae consisting of G1, G2, G3, G4, G5, G6, G7, G8,
G9 and G10 as provided below:

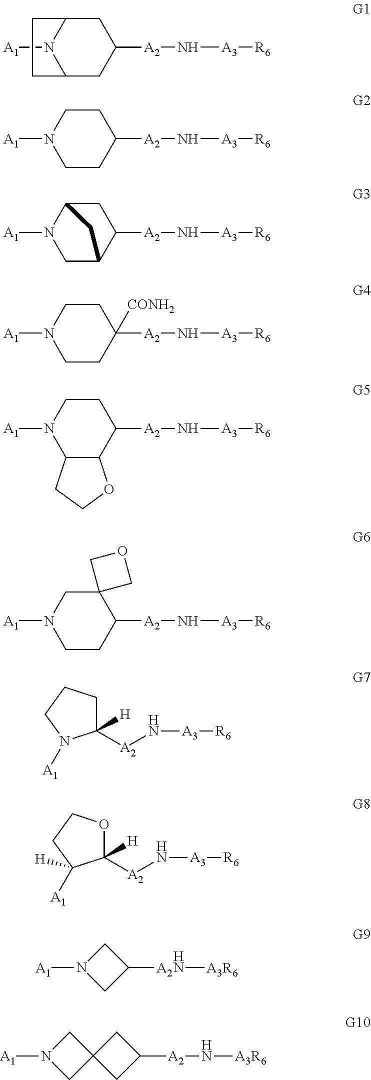

A₂ is CR₄R₅ or is absent; wherein R₄ and R₅ are each independently hydrogen or
C₁₋₆ alkyl; A₃ is —CH₂—, C(═O) or SO₂; wherein, R₆ is selected from a group consisting of a
i) substituted or unsubstituted monocylic or bicyclic aryl;
ii) substituted or unsubstituted monocylic or bicyclic heteroaryl;
iii) monocyclic aryl and hetero-aryl can be five or six membered ring bearing one or two hetero atom (N, O, S)
iv) aryl or hetero aryl ring substituted independently with halogen (F, Cl, Br), NO₂, CN, OMe, Me, CF₃, OCF₃, Ethyl, Butyl, isobutyl, small alkyl chain substituted with halogen, amino, NMe₂ alkoxy, carbonyl or sulfonyl.
v) monocyclic or bicyclic aryl or heteroaryl is fused to saturated or unsaturated cyclic ring containing at least one heteroatom selected from the group consisting of oxygen, nitrogen and sulphur which is optionally substituted with halogen, C₁₋₆ alkyl, C₁₋₆ alkoxy, C₁₋₆ alkyl substituted with C₁₋₆ alkoxy, C₁₋₆ alkoxy optionally substituted with C₁₋₆ alkyl, C₁₄ haloalkoxy, C₁₄ haloalkyl, C₁₄ thioalkyl, nitro, cyano, carboxy, C₁₄ alkylsulfonyl, aminosulfonyl, hydroxyl, amino, aminoalkyl, oxo, hydroxyalkyl, alkynyl, alkylcarbonyl and carbonyl.

### [1] A Compound of Formula (I) Selected from the Group Consisting of

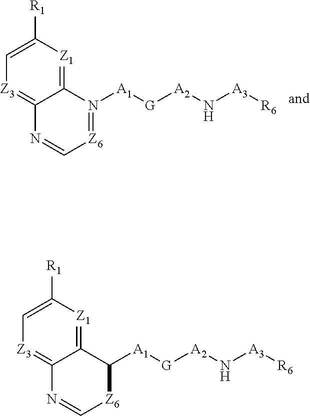

or a pharmaceutically acceptable salt thereof, wherein,
Z₁ and Z₃ are each independently selected from a group consisting of CH or N;
Z₆ is independently selected from group consisting of CH, C—CN, C═O, wherein the bold line is an optional double bond;
R₁ is independently selected from the group consisting of hydrogen, methoxy, cyano, halogen, hydroxyl, C₁₋₆ alkoxy and C₁₋₆ alkyl optionally substituted with one or two C₁₋₆alkoxy;
A₁-G-A₂-NH-A₃-R₆ is selected from the group of formulae consisting of G1, G2, G3, G4, G5, G6, G7, G8, G9 and G10 as provided below

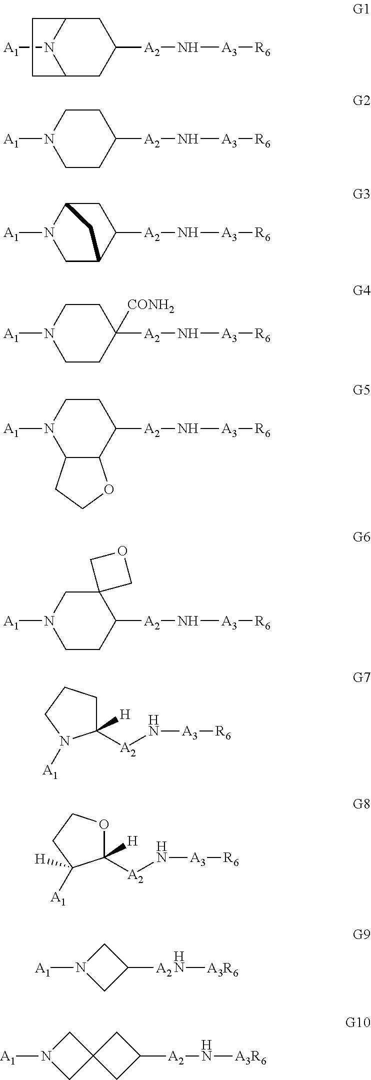

wherein, A₁ is selected from the group consisting of —(CR₂R₃)ₘ—CH₂—, —CH₂—(CR₂R₃)ₘ—, —NH—(CR₂R₃)ₘ—CH₂, —(CR₂R₃)ₘ—CH₂—O— and

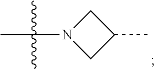

Wherein, when Z6 is C—CN and A1-G-A2-NH-A3-R6 is G2, A1 is

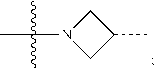

m is 1 or 2;
————— is connectivity to G;
R₂ is selected from the group consisting of hydrogen, halogen, acyloxy, C₁₋₆ alkyl optionally substituted with one or two C₁₋₆alkoxy and C₁₋₆alkoxy optionally substituted with C₁₋₆ alkyl;
R₃ is hydrogen;
A₂ is CR₄R₅ or is absent; wherein R₄ and R₅ are each independently hydrogen or C₁₋₆ alkyl;
A₃ is —CH₂—, C(═O) or SO₂; and
wherein, R₆ is selected from the group consisting of
(i) a substituted or unsubstituted monocylic or substituted or unsubstituted bicyclic aryl; and
(ii) a substituted or unsubstituted monocylic or substituted or
Unsubstituted bicyclic heteroaryl.

In one aspect, VT-03 compounds of the invention show minimal (insignificant) hERG binding activity indicating the advantage of these compounds as against the known compounds in the art (BMCL, 21, 7489-7495, 2011). VT-03 compounds of formula I of the invention are useful in the treatment of patients suffering from infections caused by Staphylococcus species, Enterococcus species, Streptococcus species, Moraxella species, E. coli, Klebsiella species, Pseudomonas species and Acinetobacter species.

In another aspect, the invention also provides compounds of formula (II) or pharmaceutically acceptable salts thereof:

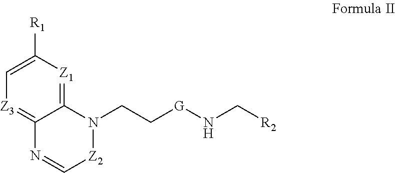

wherein, Z₁ and Z₃ are each independently selected from a group consisting of CH or N;
Z₂ is independently selected from group consisting of C═O and C═S;
R₁ is independently selected from the group consisting of hydrogen, methoxy, cyano, halogen, hydroxyl, C₁₋₆ alkoxy and C₁₋₆ alkyl optionally substituted with one or two C₁₋₆ alkoxy, alkyne, carboxyl, carboxamide;

### G is G1, G2 or G3

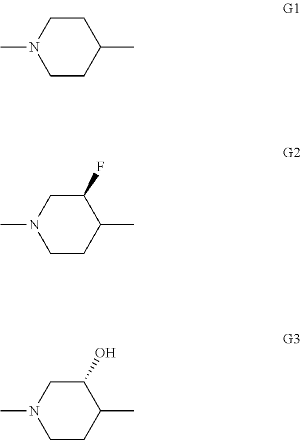

wherein, R₂ is selected from the group consisting of
(i) a substituted or unsubstituted monocylic aryl; and
(ii) a substituted or unsubstituted monocylic heteroaryl.

VT-03 compounds of Formula II of the invention are useful in the treatment of patients suffering from infections caused by Staphylococcus species, Enterococcus species, Streptococcus species, Moraxella species, E. coli, Neisseria meningitidis, Neisseria gonorrhoeae, Klebsiella species, Pseudomonas species and Acinetobacter species.

### [2] In an Embodiment the Instant Invention Provides Preferred VT-03 Compounds of Formula I in Table I

TABLE I
VT-03 Compounds of the Invention

| | |
|---|---|
| 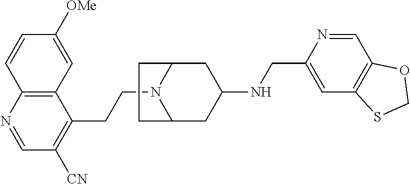 [SMILES: COc1ccc2ncc(C#N)c(CCN3C4CCC3CC(NCc3cc5c(cn3)OCS5)C4)c2c1] | VT-03-00014 |
| 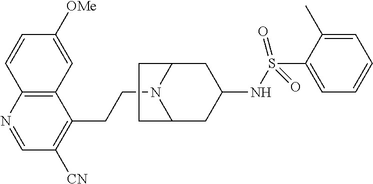 [SMILES: COc1ccc2ncc(C#N)c(CCN3C4CCC3CC(NS(=O)(=O)c3ccccc3C)C4)c2c1] | VT-03-00017 |
| 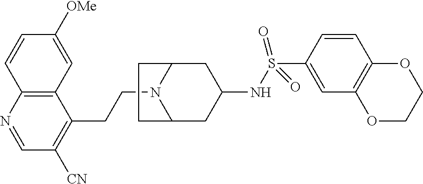 [SMILES: COc1ccc2ncc(C#N)c(CCN3C4CCC3CC(NS(=O)(=O)c3ccc5c(c3)OCCO5)C4)c2c1] | VT-03-00021 |
| 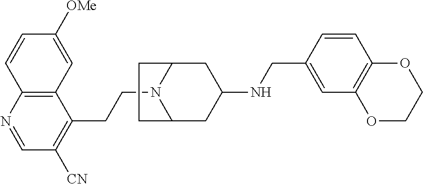 [SMILES: COc1ccc2ncc(C#N)c(CCN3C4CCC3CC(NCc3ccc5c(c3)OCCO5)C4)c2c1] | VT-03-00021a |
| 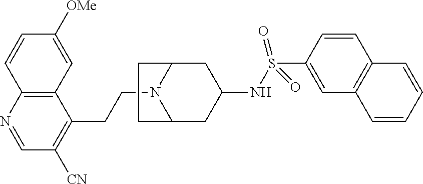 [SMILES: COc1ccc2ncc(C#N)c(CCN3C4CCC3CC(NS(=O)(=O)c3ccc5ccccc5c3)C4)c2c1] | VT-03-00022 |
| 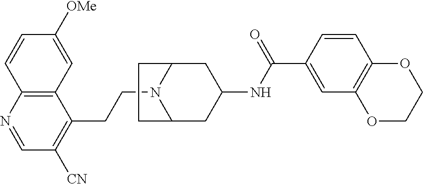 [SMILES: COc1ccc2ncc(C#N)c(CCN3C4CCC3CC(NC(=O)c3ccc5c(c3)OCCO5)C4)c2c1] | VT-03-00024 |
| 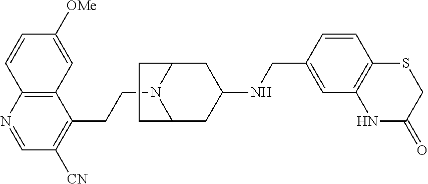 [SMILES: COc1ccc2ncc(C#N)c(CCN3C4CCC3CC(NCc3ccc5c(c3)NC(=O)CS5)C4)c2c1] | VT-03-00026 |
| 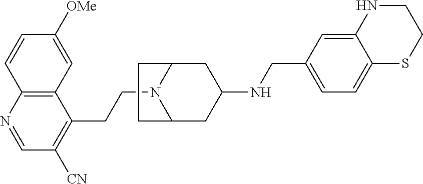 [SMILES: COc1ccc2ncc(C#N)c(CCN3C4CCC3CC(NCc3ccc5c(c3)NCCS5)C4)c2c1] | VT-03-00026a |
| 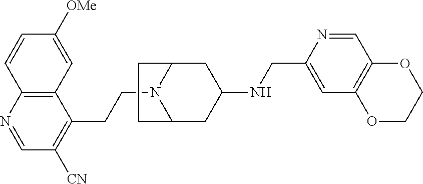 [SMILES: COc1ccc2ncc(C#N)c(CCN3C4CCC3CC(NCc3cc5c(cn3)OCCO5)C4)c2c1] | VT-03-00027 |
| 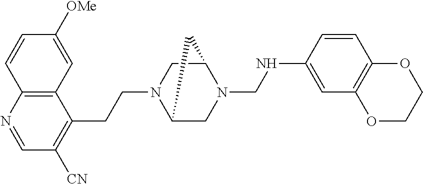 [SMILES: COc1ccc2ncc(C#N)c(CCN3C[C@@H]4C[C@H]3CN4CNc3ccc4c(c3)OCCO4)c2c1] | VT-03-00028 |
| 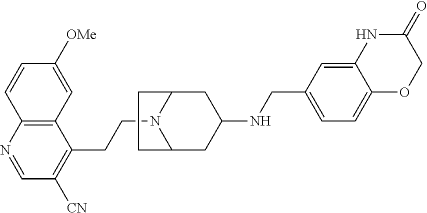 [SMILES: COc1ccc2ncc(C#N)c(CCN3C4CCC3CC(NCc3ccc5c(c3)NC(=O)CO5)C4)c2c1] | VT-03-00030 |
| 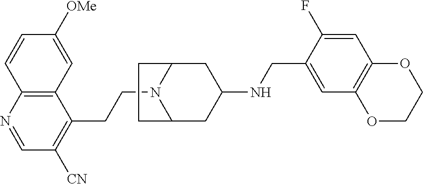 [SMILES: COc1ccc2ncc(C#N)c(CCN3C4CCC3CC(NCc3cc5c(cc3F)OCCO5)C4)c2c1] | VT-03-00031 |
| 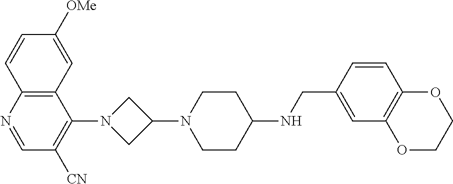 [SMILES: COc1ccc2ncc(C#N)c(N3CC(N4CCC(NCc5ccc6c(c5)OCCO6)CC4)C3)c2c1] | VT-03-00032 |
| 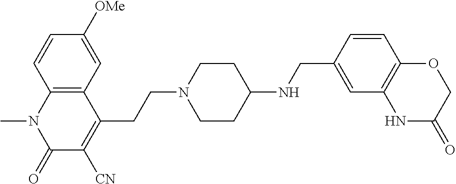 [SMILES: COc1ccc2c(c1)c(CCN1CCC(NCc3ccc4c(c3)NC(=O)CO4)CC1)c(C#N)c(=O)n2C] | VT-03-00042 |
| 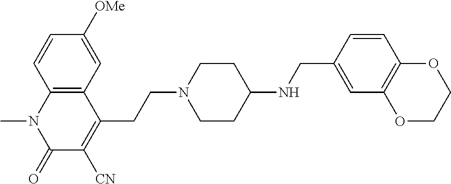 [SMILES: COc1ccc2c(c1)c(CCN1CCC(NCc3ccc4c(c3)OCCO4)CC1)c(C#N)c(=O)n2C] | VT-03-00043 |
| 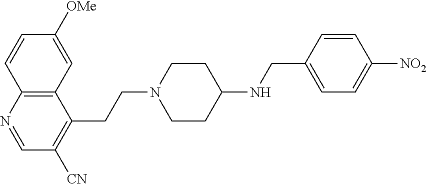 [SMILES: COc1ccc2ncc(C#N)c(CCN3CCC(NCc4ccc([N+](=O)[O-])cc4)CC3)c2c1] | VT-03-00045 |
| 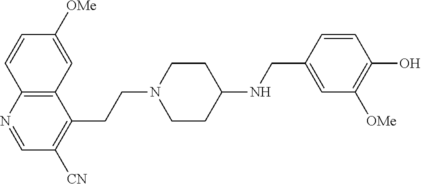 [SMILES: COc1ccc2ncc(C#N)c(CCN3CCC(NCc4ccc(O)c(OC)c4)CC3)c2c1] | VT-03-00046 |
| 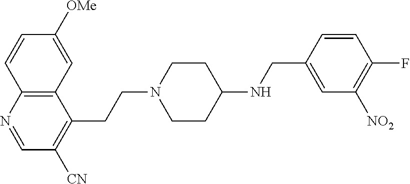 [SMILES: COc1ccc2ncc(C#N)c(CCN3CCC(NCc4ccc(F)c([N+](=O)[O-])c4)CC3)c2c1] | VT-03-00048 |
| 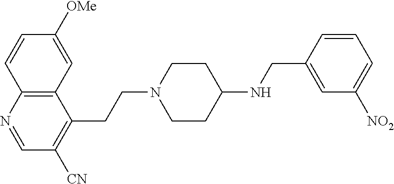 [SMILES: COc1ccc2ncc(C#N)c(CCN3CCC(NCc4cccc([N+](=O)[O-])c4)CC3)c2c1] | VT-03-00049 |
| 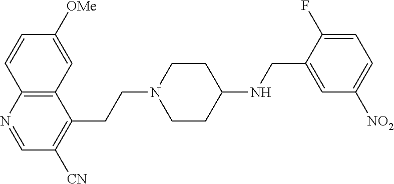 [SMILES: COc1ccc2ncc(C#N)c(CCN3CCC(NCc4cc([N+](=O)[O-])ccc4F)CC3)c2c1] | VT-03-00050 |
| 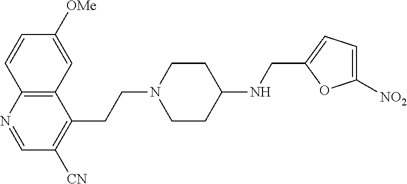 [SMILES: COc1ccc2ncc(C#N)c(CCN3CCC(NCc4ccc([N+](=O)[O-])o4)CC3)c2c1] | VT-03-00051 |
| 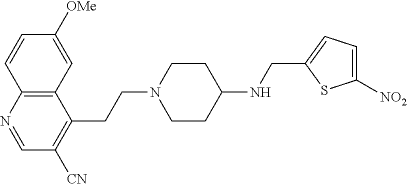 [SMILES: COc1ccc2ncc(C#N)c(CCN3CCC(NCc4ccc([N+](=O)[O-])s4)CC3)c2c1] | VT-03-00052 |
| 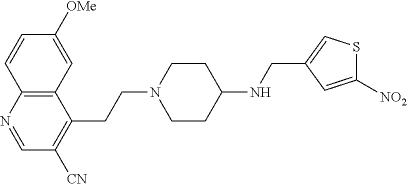 [SMILES: COc1ccc2ncc(C#N)c(CCN3CCC(NCc4csc([N+](=O)[O-])c4)CC3)c2c1] | VT-03-00053 |
| 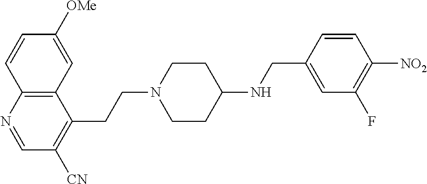 [SMILES: COc1ccc2ncc(C#N)c(CCN3CCC(NCc4ccc([N+](=O)[O-])c(F)c4)CC3)c2c1] | VT-03-00054 |
| 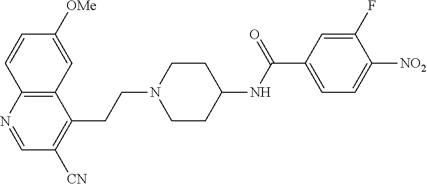 [SMILES: COc1ccc2ncc(C#N)c(CCN3CCC(NC(=O)c4ccc([N+](=O)[O-])c(F)c4)CC3)c2c1] | VT-03-00055 |
| 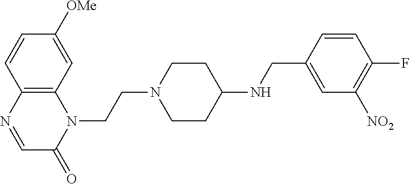 [SMILES: COc1ccc2ncc(=O)n(CCN3CCC(NCc4ccc(F)c([N+](=O)[O-])c4)CC3)c2c1] | VT-03-00056 |
| 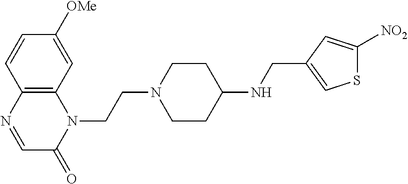 [SMILES: COc1ccc2ncc(=O)n(CCN3CCC(NCc4csc([N+](=O)[O-])c4)CC3)c2c1] | VT-03-00057 |
| 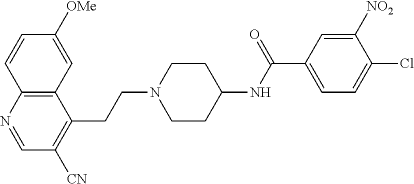 [SMILES: COc1ccc2ncc(C#N)c(CCN3CCC(NC(=O)c4ccc(Cl)c([N+](=O)[O-])c4)CC3)c2c1] | VT-03-00058 |
| 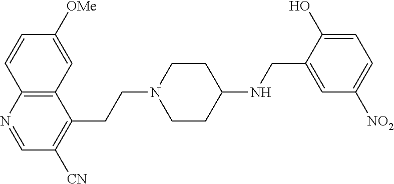 [SMILES: COc1ccc2ncc(C#N)c(CCN3CCC(NCc4cc([N+](=O)[O-])ccc4O)CC3)c2c1] | VT-03-00059 |
| 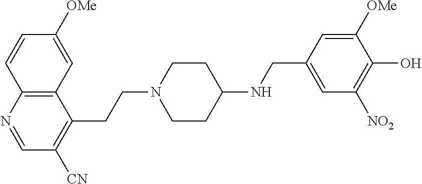 [SMILES: COc1ccc2ncc(C#N)c(CCN3CCC(NCc4cc(OC)c(O)c([N+](=O)[O-])c4)CC3)c2c1] | VT-03-00060 |
| 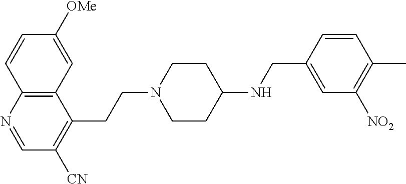 [SMILES: COc1ccc2ncc(C#N)c(CCN3CCC(NCc4ccc(C)c([N+](=O)[O-])c4)CC3)c2c1] | VT-03-00061 |
| 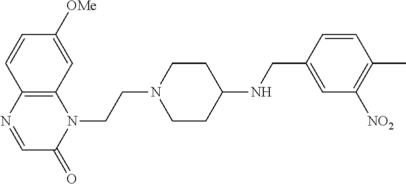 [SMILES: COc1ccc2ncc(=O)n(CCN3CCC(NCc4ccc(C)c([N+](=O)[O-])c4)CC3)c2c1] | VT-03-00062 |
| 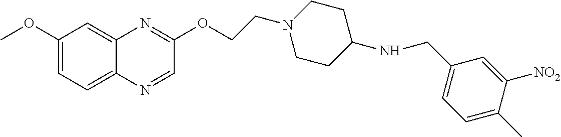 [SMILES: COc1ccc2ncc(OCCN3CCC(NCc4ccc(C)c([N+](=O)[O-])c4)CC3)nc2c1] | VT-03-00062a |
| 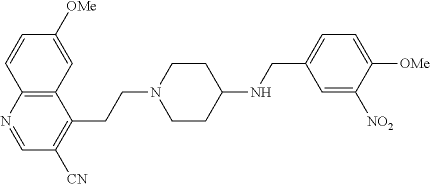 [SMILES: COc1ccc2ncc(C#N)c(CCN3CCC(NCc4ccc(OC)c([N+](=O)[O-])c4)CC3)c2c1] | VT-03-00063 |
| 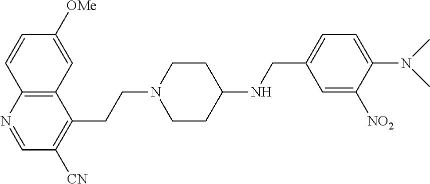 [SMILES: COc1ccc2ncc(C#N)c(CCN3CCC(NCc4ccc(N(C)C)c([N+](=O)[O-])c4)CC3)c2c1] | VT-03-00064 |
| 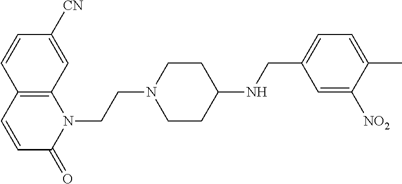 [SMILES: Cc1ccc(CNC2CCN(CCn3c(=O)ccc4ccc(C#N)cc43)CC2)cc1[N+](=O)[O-]] | VT-03-00065 |
| 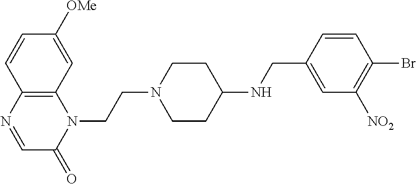 [SMILES: COc1ccc2ncc(=O)n(CCN3CCC(NCc4ccc(Br)c([N+](=O)[O-])c4)CC3)c2c1] | VT-03-00066 |
| 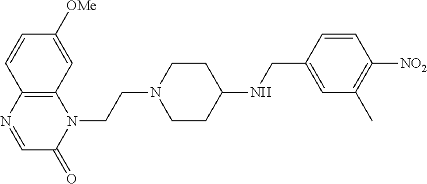 [SMILES: COc1ccc2ncc(=O)n(CCN3CCC(NCc4ccc([N+](=O)[O-])c(C)c4)CC3)c2c1] | VT-03-00067 |
| 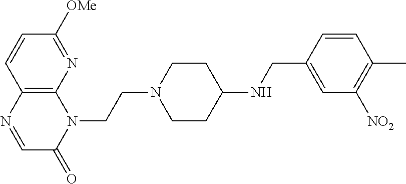 [SMILES: COc1ccc2ncc(=O)n(CCN3CCC(NCc4ccc(C)c([N+](=O)[O-])c4)CC3)c2n1] | VT-03-00068 |
| 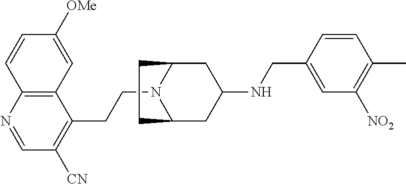 [SMILES: COc1ccc2ncc(C#N)c(CCN3[C@@H]4CC[C@H]3CC(NCc3ccc(C)c([N+](=O)[O-])c3)C4)c2c1] | VT-03-00069 |
| 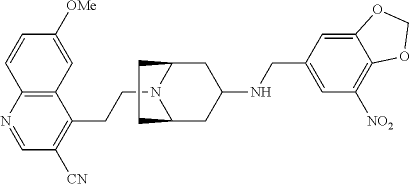 [SMILES: COc1ccc2ncc(C#N)c(CCN3[C@@H]4CC[C@H]3CC(NCc3cc5c(c([N+](=O)[O-])c3)OCO5)C4)c2c1] | VT-03-00070 |
| 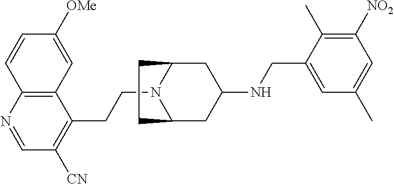 [SMILES: COc1ccc2ncc(C#N)c(CCN3[C@@H]4CC[C@H]3CC(NCc3cc(C)cc([N+](=O)[O-])c3C)C4)c2c1] | VT-03-00071 |
| 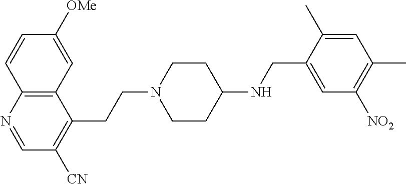 [SMILES: COc1ccc2ncc(C#N)c(CCN3CCC(NCc4cc([N+](=O)[O-])c(C)cc4C)CC3)c2c1] | VT-03-00072 |
| 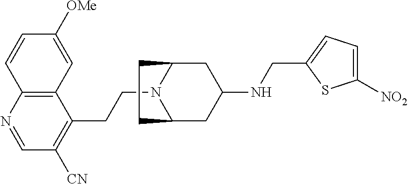 [SMILES: COc1ccc2ncc(C#N)c(CCN3[C@@H]4CC[C@H]3CC(NCc3ccc([N+](=O)[O-])s3)C4)c2c1] | VT-03-00074 |
| 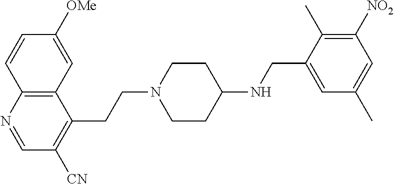 [SMILES: COc1ccc2ncc(C#N)c(CCN3CCC(NCc4cc(C)cc([N+](=O)[O-])c4C)CC3)c2c1] | VT-03-00075 |
| 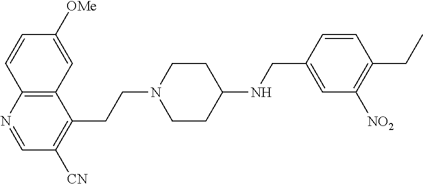 [SMILES: CCc1ccc(CNC2CCN(CCc3c(C#N)cnc4ccc(OC)cc34)CC2)cc1[N+](=O)[O-]] | VT-03-00076 |
| 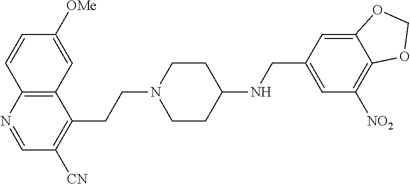 [SMILES: COc1ccc2ncc(C#N)c(CCN3CCC(NCc4cc5c(c([N+](=O)[O-])c4)OCO5)CC3)c2c1] | VT-03-00077 |
| 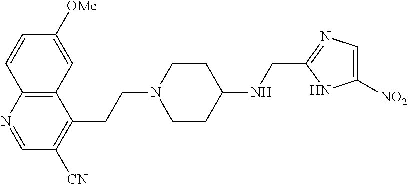 [SMILES: COc1ccc2ncc(C#N)c(CCN3CCC(NCc4ncc([N+](=O)[O-])[nH]4)CC3)c2c1] | VT-03-00078 |
| 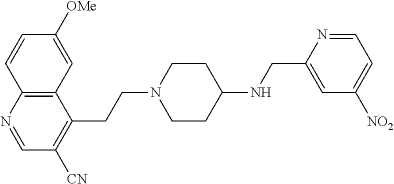 [SMILES: COc1ccc2ncc(C#N)c(CCN3CCC(NCc4cc([N+](=O)[O-])ccn4)CC3)c2c1] | VT-03-00079 |
| 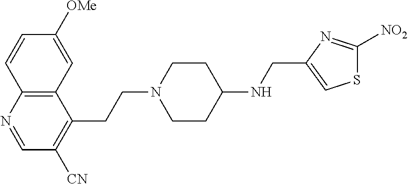 [SMILES: COc1ccc2ncc(C#N)c(CCN3CCC(NCc4csc([N+](=O)[O-])n4)CC3)c2c1] | VT-03-00080 |
| 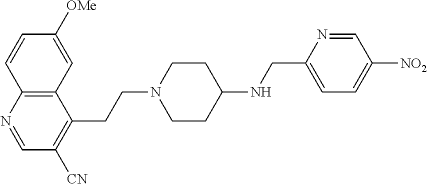 [SMILES: COc1ccc2ncc(C#N)c(CCN3CCC(NCc4ccc([N+](=O)[O-])cn4)CC3)c2c1] | VT-03-00081 |
| 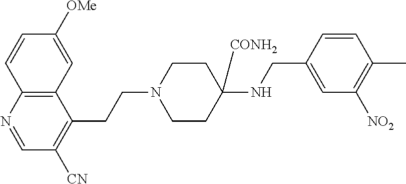 [SMILES: COc1ccc2ncc(C#N)c(CCN3CCC(NCc4ccc(C)c([N+](=O)[O-])c4)(C(N)=O)CC3)c2c1] | VT-03-00083 |
| 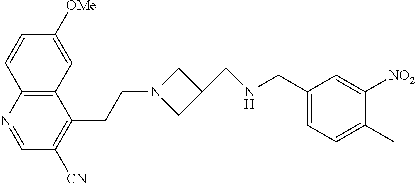 [SMILES: COc1ccc2ncc(C#N)c(CCN3CC(CNCc4ccc(C)c([N+](=O)[O-])c4)C3)c2c1] | VT-03-00084 |
| 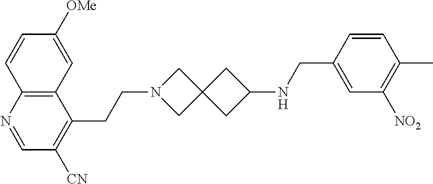 [SMILES: COc1ccc2ncc(C#N)c(CCN3CC4(CC(NCc5ccc(C)c([N+](=O)[O-])c5)C4)C3)c2c1] | VT-03-00085 |
| 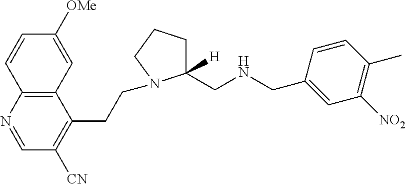 [SMILES: COc1ccc2ncc(C#N)c(CCN3CCC[C@@H]3CNCc3ccc(C)c([N+](=O)[O-])c3)c2c1] | VT-03-00086 |
| 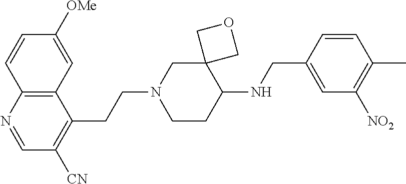 [SMILES: COc1ccc2ncc(C#N)c(CCN3CCC(NCc4ccc(C)c([N+](=O)[O-])c4)C4(COC4)C3)c2c1] | VT-03-00087 |
| 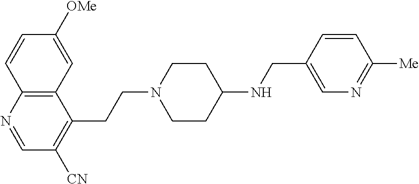 [SMILES: COc1ccc2ncc(C#N)c(CCN3CCC(NCc4ccc(C)nc4)CC3)c2c1] | VT-03-00088 |
| 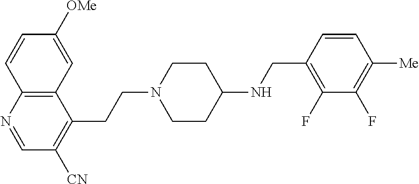 [SMILES: COc1ccc2ncc(C#N)c(CCN3CCC(NCc4ccc(C)c(F)c4F)CC3)c2c1] | VT-03-00089 |
| 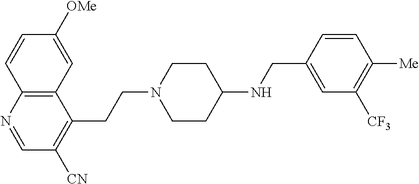 [SMILES: COc1ccc2ncc(C#N)c(CCN3CCC(NCc4ccc(C)c(C(F)(F)F)c4)CC3)c2c1] | VT-03-00090 |
| 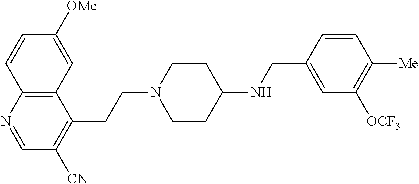 [SMILES: COc1ccc2ncc(C#N)c(CCN3CCC(NCc4ccc(C)c(OC(F)(F)F)c4)CC3)c2c1] | VT-03-00091 |
| 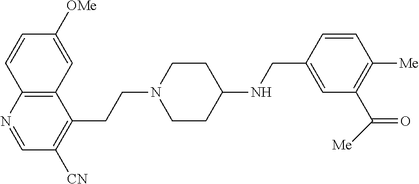 [SMILES: COc1ccc2ncc(C#N)c(CCN3CCC(NCc4ccc(C)c(C(C)=O)c4)CC3)c2c1] | VT-03-00092 |
| 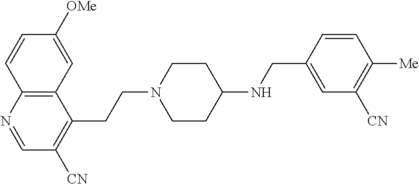 [SMILES: COc1ccc2ncc(C#N)c(CCN3CCC(NCc4ccc(C)c(C#N)c4)CC3)c2c1] | VT-03-00093 |
| 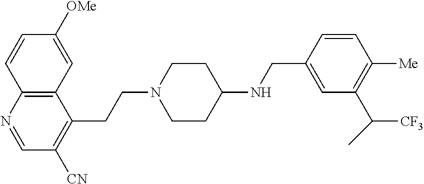 [SMILES: COc1ccc2ncc(C#N)c(CCN3CCC(NCc4ccc(C)c(C(C)C(F)(F)F)c4)CC3)c2c1] | VT-03-00094 |
| 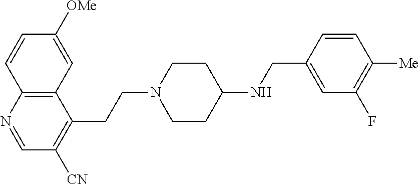 [SMILES: COc1ccc2ncc(C#N)c(CCN3CCC(NCc4ccc(C)c(F)c4)CC3)c2c1] | VT-03-00095 |

In another embodiment the instant invention provides preferred VT-03 compounds of formula II as in Table II

TABLE II
VT-03 Compounds of the Invention

| | |
|---|---|
| 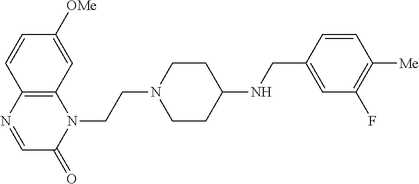 [SMILES: COc1ccc2ncc(=O)n(CCN3CCC(NCc4ccc(C)c(F)c4)CC3)c2c1] | VT-03-00100 |
| 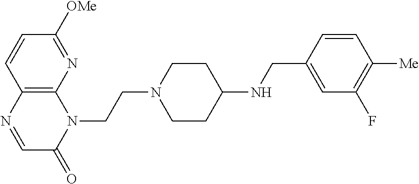 [SMILES: COc1ccc2ncc(=O)n(CCN3CCC(NCc4ccc(C)c(F)c4)CC3)c2n1] | VT-03-00101 |
| 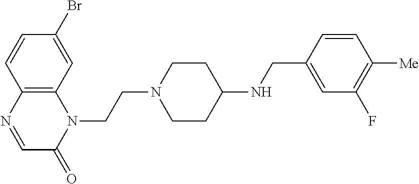 [SMILES: Cc1ccc(CNC2CCN(CCn3c(=O)cnc4ccc(Br)cc43)CC2)cc1F] | VT-03-00102 |
| 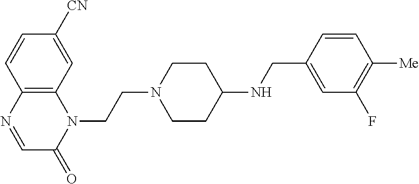 [SMILES: Cc1ccc(CNC2CCN(CCn3c(=O)cnc4ccc(C#N)cc43)CC2)cc1F] | VT-03-00103 |
| 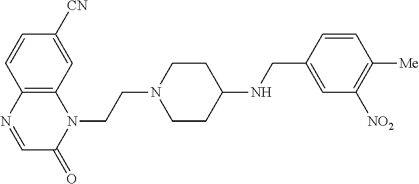 [SMILES: Cc1ccc(CNC2CCN(CCn3c(=O)cnc4ccc(C#N)cc43)CC2)cc1[N+](=O)[O-]] | VT-03-00104 |
| 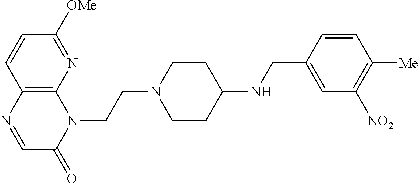 [SMILES: COc1ccc2ncc(=O)n(CCN3CCC(NCc4ccc(C)c([N+](=O)[O-])c4)CC3)c2n1] | VT-03-00106 |
| 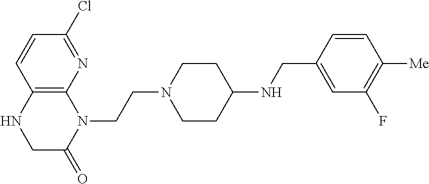 [SMILES: Cc1ccc(CNC2CCN(CCN3C(=O)CNc4ccc(Cl)nc43)CC2)cc1F] | VT-03-00107 |
| 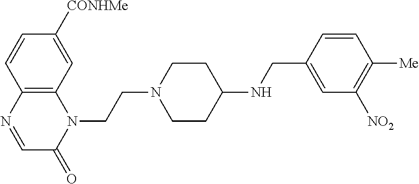 | VT-03-00108 |
| 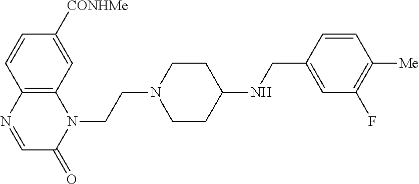 | VT-03-00109 |
| 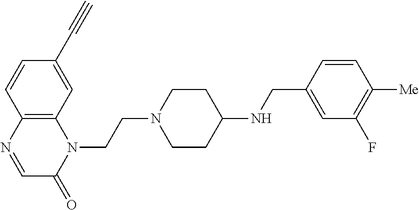 [SMILES: C#Cc1ccc2ncc(=O)n(CCN3CCC(NCc4ccc(C)c(F)c4)CC3)c2c1] | VT-03-00110 |
| 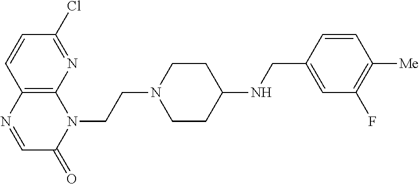 [SMILES: Cc1ccc(CNC2CCN(CCn3c(=O)cnc4ccc(Cl)nc43)CC2)cc1F] | VT-03-00111 |
| 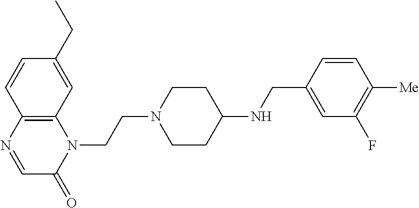 [SMILES: CCc1ccc2ncc(=O)n(CCN3CCC(NCc4ccc(C)c(F)c4)CC3)c2c1] | VT-03-00112 |
| 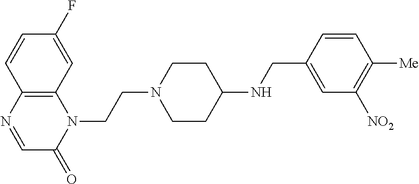 [SMILES: Cc1ccc(CNC2CCN(CCn3c(=O)cnc4ccc(F)cc43)CC2)cc1[N+](=O)[O-]] | VT-03-00113 |
| 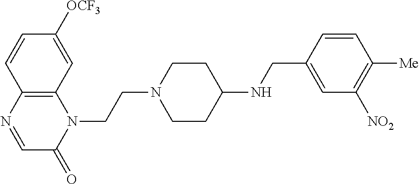 [SMILES: Cc1ccc(CNC2CCN(CCn3c(=O)cnc4ccc(OC(F)(F)F)cc43)CC2)cc1[N+](=O)[O-]] | VT-03-00114 |
| 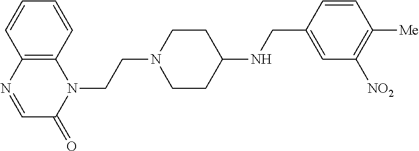 [SMILES: Cc1ccc(CNC2CCN(CCn3c(=O)cnc4ccccc43)CC2)cc1[N+](=O)[O-]] | VT-03-00115 |
| 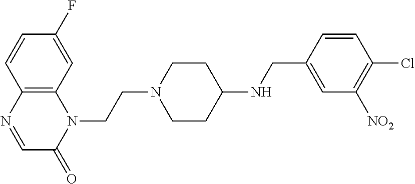 [SMILES: O=c1cnc2ccc(F)cc2n1CCN1CCC(NCc2ccc(Cl)c([N+](=O)[O-])c2)CC1] | VT-03-00116 |
| 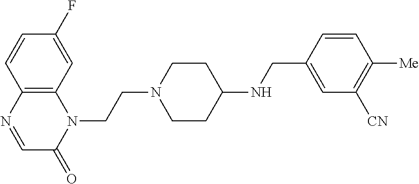 [SMILES: Cc1ccc(CNC2CCN(CCn3c(=O)cnc4ccc(F)cc43)CC2)cc1C#N] | VT-03-00117 |
| 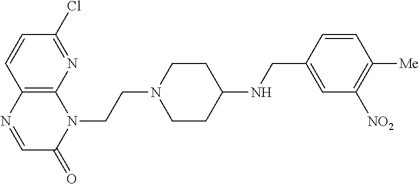 [SMILES: Cc1ccc(CNC2CCN(CCn3c(=O)cnc4ccc(Cl)nc43)CC2)cc1[N+](=O)[O-]] | VT-03-00118 |
| 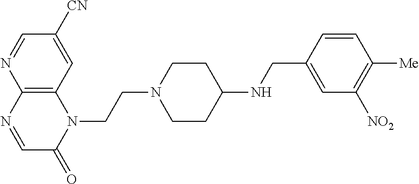 [SMILES: Cc1ccc(CNC2CCN(CCn3c(=O)cnc4ncc(C#N)cc43)CC2)cc1[N+](=O)[O-]] | VT-03-00119 |
| 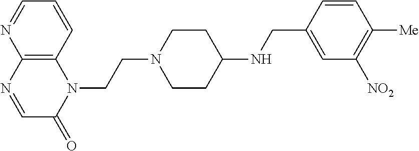 [SMILES: Cc1ccc(CNC2CCN(CCn3c(=O)cnc4ncccc43)CC2)cc1[N+](=O)[O-]] | VT-03-00120 |
| 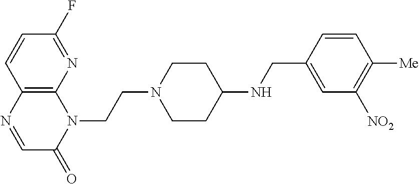 [SMILES: Cc1ccc(CNC2CCN(CCn3c(=O)cnc4ccc(F)nc43)CC2)cc1[N+](=O)[O-]] | VT-03-00121 |
| 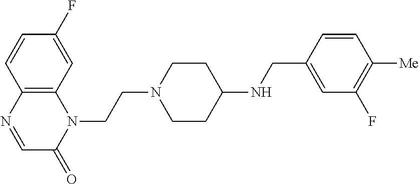 [SMILES: Cc1ccc(CNC2CCN(CCn3c(=O)cnc4ccc(F)cc43)CC2)cc1F] | VT-03-00122 |
| 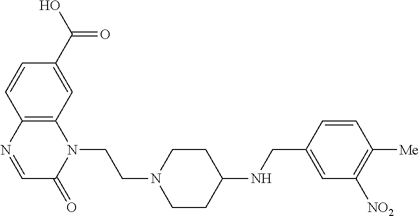 [SMILES: Cc1ccc(CNC2CCN(CCn3c(=O)cnc4ccc(C(=O)O)cc43)CC2)cc1[N+](=O)[O-]] | VT-03-00124 |
| 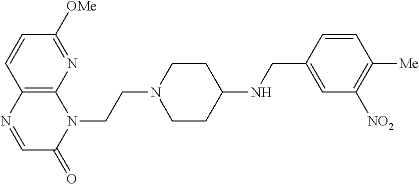 [SMILES: COc1ccc2ncc(=O)n(CCN3CCC(NCc4ccc(C)c([N+](=O)[O-])c4)CC3)c2n1] | VT-03-00125 |
| 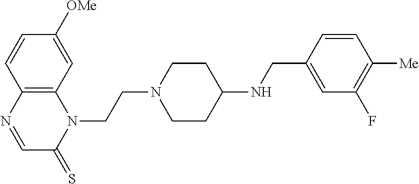 [SMILES: COc1ccc2ncc(=S)n(CCN3CCC(NCc4ccc(C)c(F)c4)CC3)c2c1] | VT-03-00126 |
| 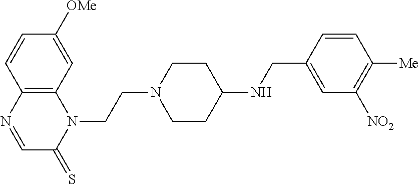 [SMILES: COc1ccc2ncc(=S)n(CCN3CCC(NCc4ccc(C)c([N+](=O)[O-])c4)CC3)c2c1] | VT-03-00127 |
| 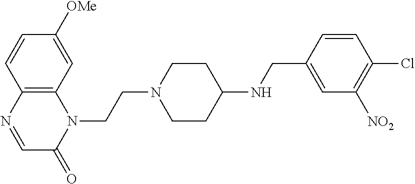 [SMILES: COc1ccc2ncc(=O)n(CCN3CCC(NCc4ccc(Cl)c([N+](=O)[O-])c4)CC3)c2c1] | VT-03-00128 |
| 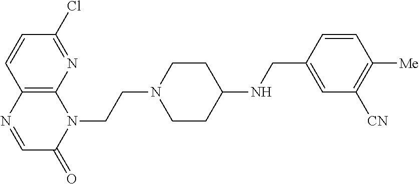 [SMILES: Cc1ccc(CNC2CCN(CCn3c(=O)cnc4ccc(Cl)nc43)CC2)cc1C#N] | VT-03-00131 |
| 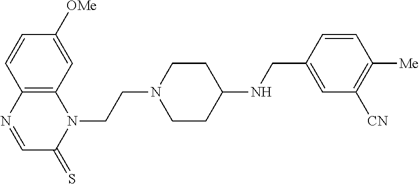 [SMILES: COc1ccc2ncc(=S)n(CCN3CCC(NCc4ccc(C)c(C#N)c4)CC3)c2c1] | VT-03-00132 |
| 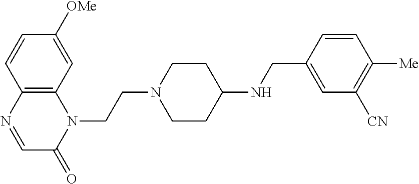 [SMILES: COc1ccc2ncc(=O)n(CCN3CCC(NCc4ccc(C)c(C#N)c4)CC3)c2c1] | VT-03-00133 |
| 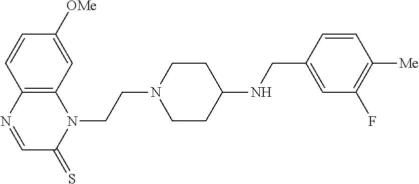 [SMILES: COc1ccc2ncc(=S)n(CCN3CCC(NCc4ccc(C)c(F)c4)CC3)c2c1] | VT-03-00126 |
| 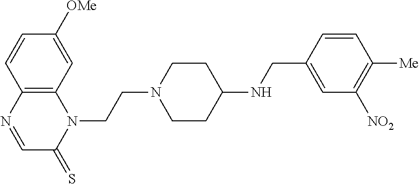 [SMILES: COc1ccc2ncc(=S)n(CCN3CCC(NCc4ccc(C)c([N+](=O)[O-])c4)CC3)c2c1] | VT-03-00127 |
| 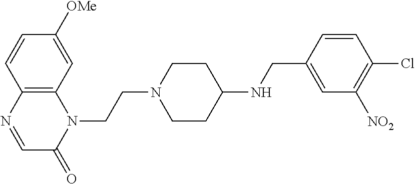 [SMILES: COc1ccc2ncc(=O)n(CCN3CCC(NCc4ccc(Cl)c([N+](=O)[O-])c4)CC3)c2c1] | VT-03-00128 |
| 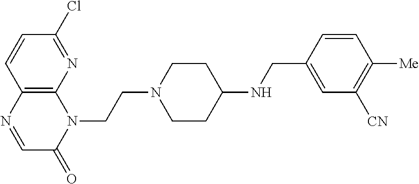 [SMILES: Cc1ccc(CNC2CCN(CCn3c(=O)cnc4ccc(Cl)nc43)CC2)cc1C#N] | VT-03-00131 |
| 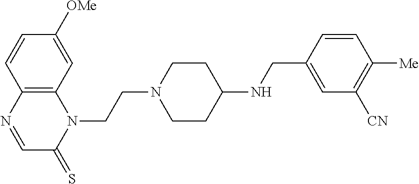 [SMILES: COc1ccc2ncc(=S)n(CCN3CCC(NCc4ccc(C)c(C#N)c4)CC3)c2c1] | VT-03-00132 |
| 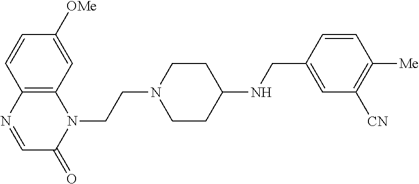 [SMILES: COc1ccc2ncc(=O)n(CCN3CCC(NCc4ccc(C)c(C#N)c4)CC3)c2c1] | VT-03-00133 |
| 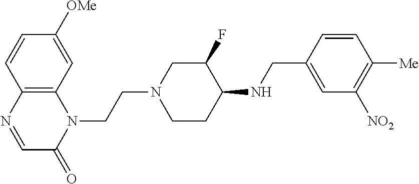 [SMILES: COc1ccc2ncc(=O)n(CCN3CC[C@H](NCc4ccc(C)c([N+](=O)[O-])c4)[C@H](F)C3)c2c1] | VT-03-00134 |
| 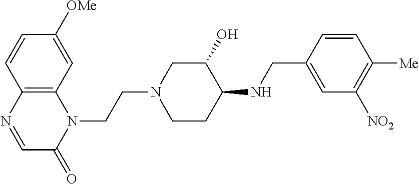 [SMILES: COc1ccc2ncc(=O)n(CCN3CC[C@H](NCc4ccc(C)c([N+](=O)[O-])c4)[C@@H](O)C3)c2c1] | VT-03-00135 |

The invention further consists of the following:

### [3] General Synthesis of VT-03 Compounds of the Invention

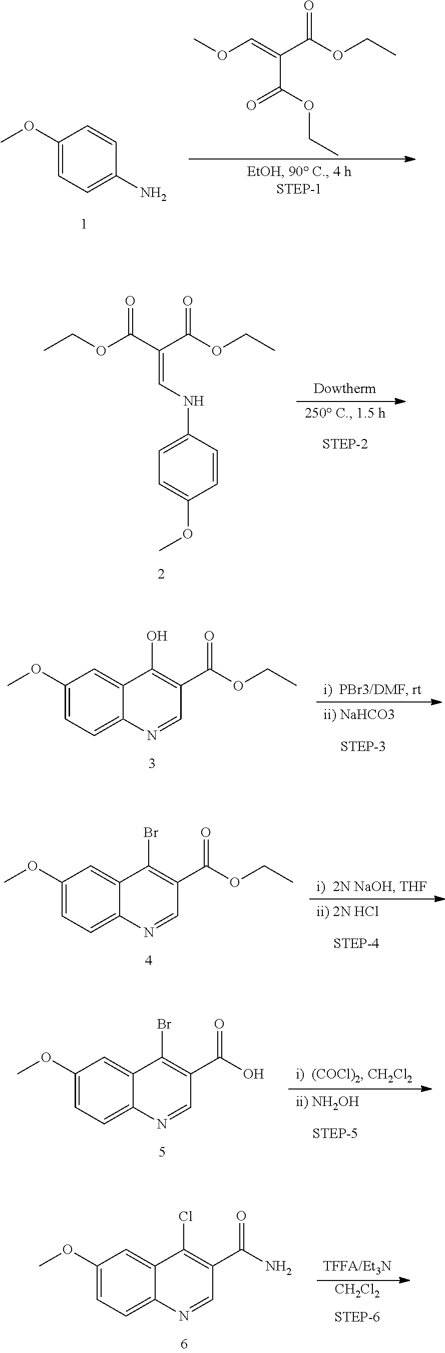

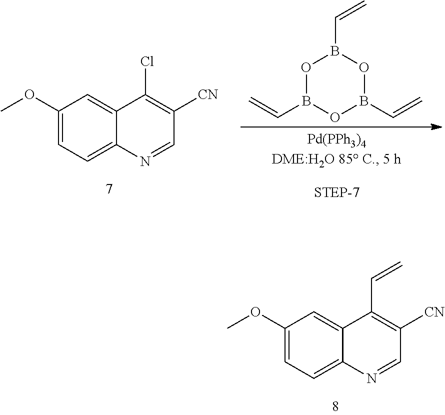

### Preparation of 6-Methoxy-4-vinyl-quinoline-3-carbonitrile (Compound of Step 8)

### a) 2-[(4-Methoxy-phenylamino)-methylene]-malonic acid diethyl ester

To a solution of 4-aminoanisole (40 g, 324.8 mmol) in ethanol was added diethyl ethoxymethylenemalonate (70.23 g, 324.8 mmol). The reaction was refluxed at 85° C. for 4 h. The ethanol in the reaction mixture was distilled out under reduced pressure. The residue was chromatographed on silicagel eluting with 5% ethylacetate in hexane to afford the product as oil 2 (61 g).

### b) 4-Hydroxy-6-methoxy-quinoline-3-carboxylic acid ethyl ester

Compound 2 (61 g) was taken up in dowtherm (400 ml) and heated at 250° C. for 3 h. The reaction mixture was cooled to (RT) and treated with pentane (300 mL) and filtered under suction. The resulting solids were washed thoroughly with excess of pentane and dried under vacuum to give 3 (23 g).

### c) 4-Bromo-6-methoxy-quinoline-3-carboxylic acid ethyl ester

To a stirred solution of compound 3 (23 g, 93 mmol) in DMF (91 mL) was added PBr₃ (8.8 mL, 93 mmol) dropwise at RT. The reaction mixture was stirred at ambient temperature for 1 h after which 200 ml ice cold water was added. The reaction was neutralized with aq. NaHCO₃ solution. The obtained solids were collected by filtration, washed with water and dried under vacuum to give the required product 4 (32 g).

### d) 4-Bromo-6-methoxy-quinoline-3-carboxylic acid

To a stirred solution of compound 4 (20 g) in THF was added 2N NaOH solution (71 mL) dropwise at 0° C. The reaction mixture was brought to RT and stirred for 24 h after which it was concentrated to remove the THF. The aqueous layer was washed with ethyl acetate to remove the insoluble impurities. The resulting aqueous layer was acidified to pH 2. The product was collected by filtration then codistilled with Toluene and dried under vacuum to afford the required compound 5 (15.5 g).

### e) 4-Chloro-6-methoxy-quinoline-3-carboxylic acid amide

To a stirred solution of compound 5 (15 g, 53.4 mmol) in anhydrous dichloromethane (200 ml) was added oxalyl chloride (9.2 mL, 106.7 mmol) dropwise at 0° C. followed by the addition of a catalytic amount of dry DMF. The reaction mixture was gradually brought to RT and stirred for 1 h. The CH₂Cl₂ and oxalyl chloride in the reaction mass were removed by distillation. The residue obtained was redissolved in CH₂Cl₂, and conc.NH₄OH solution (5 ml) was added dropwise very slowly to this solution at 0° C. (highly exothermic). The reaction was stirred for an additional 2 h. The observed solids were isolated via filtration, codistilled with Toluene and dried under vacuum to give the required compound 6 (22 g).

### f) 4-Chloro-6-methoxy-quinoline-3-carbonitrile

To a stirred solution of compound 6 (26.5 g, 112.05 mmol) in CH₂Cl₂, was added triethylamine (104 ml) at 0° C. followed by dropwise addition of trifluoroacetic anhydride (59.6 mL, 425.9 mmol) at the same temperature. The reaction was stirred at RT for 3 h and quenched by adding water (150 mL). The organic layer was separated, dried over Na₂SO₄ and concentrated under reduced pressure. The obtained residue was treated with cold hexanes and filtered under suction to afford the required compound 7 (7.5 g).

### g) 6-Methoxy-4-vinyl-quinoline-3-carbonitrile

To a stirred solution of compound 7 (3 g, 13.7 mmol) in 1,2-dimethoxyethane (90 mL) and water (30 mL) was added K₂CO₃ (14.9 mmol), Pd(PPh₃)₄ (0.274 mmol) and finally 2,4,6-trivinyl cycloborane-pyridine complex (14.9 mmol). The reaction was stirred at 80° C. for 6 h. The reaction mass was diluted with Ethyl acetate and filtered under celite. The filtrate was washed with water, dried over sodium sulfate and concentrated under reduced pressure. The obtained residue was chromatographed on silicagel eluting with 25% ethylacetate in hexanes to afford the product as a solid 8 (2.1 g).

### [3] Synthesis of Specific Compounds of the Invention

Synthesis of VT-03-00014, VT-03-00017, VT-03-00021, VT-03-00021a, VT-03-00022, VT-03-00024, VT-03-00026, VT-03-00026a, VT-03-00027, VT-03-00028, VT-03-00030, VT-03-00031, VT-03-00032, VT-03-00042, VT-03-00043, VT-03-00045, VT-03-00046, VT-03-00048, VT-03-00049, VT-03-00050, VT-03-00051, VT-03-00052, VT-03-00053, VT-03-00054, VT-03-00055, VT-03-00056, VT-03-00057, VT-03-00058, VT-03-00059, VT-03-00060, VT-03-00061, VT-03-00062, VT-03-00062A, VT-03-00063, VT-03-00064, VT-03-00065, VT-03-00066, VT-03-00067, VT-03-00068, VT-03-00069, VT-03-00070, VT-03-00071, VT-03-00072, VT-03-00074, VT-03-00075, VT-03-00076, VT-03-00077, VT-03-00078, VT-03-00079, VT-03-00080, VT-03-00081, VT-03-00083, VT-03-00084, VT-03-00085, VT-03-00086, VT-03-00087, VT-03-00088, VT-03-00089, VT-03-00090, VT-03-00091, VT-03-00092, VT-03-00093, VT-03-00094, VT-03-00095 are provided in detail in the parent U.S. application Ser. No. 14/434,870 which is incorporated herein in its entirety by reference.

It should be noted that the intermediates referred in the foregoing are described in the parent U.S. application Ser. No. 14/434,870.

### (65) Synthesis of VT-03-00101

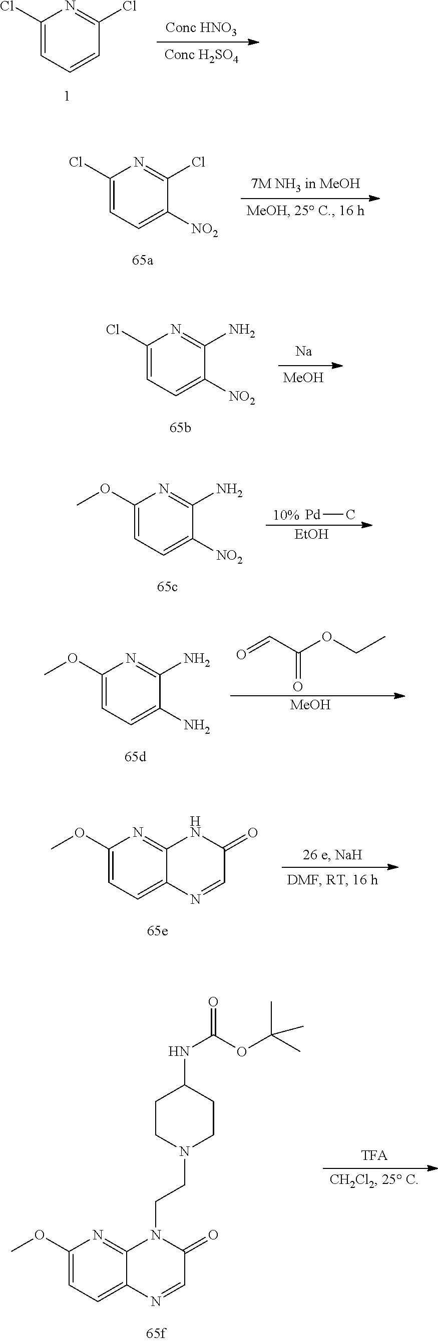

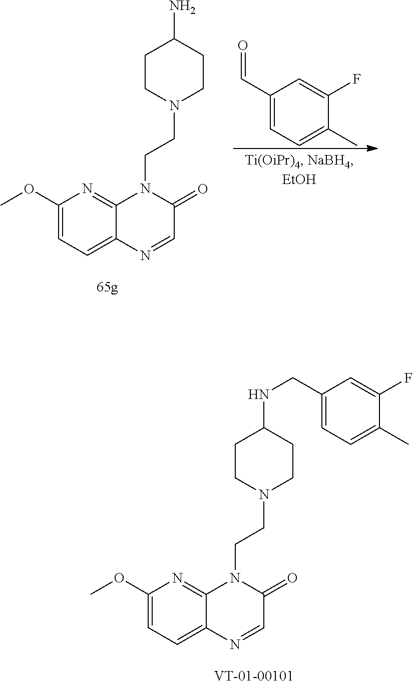

### (65a) 2,6-dichloro-3-nitropyridine

To an ice cold solution of 2,6-Dichloropyridine (25 g) in conc H₂SO₄ (60 mL), was added fuming HNO₃ (30 mL) dropwise at 0° C. This mixture was heated at 110° C. for 6 h. Upon completion the reaction was cooled to ambient temperature and poured into ice. Solids precipitated which were washed with excess water. These solids were dried to give the required compound 65a (30 g).

### (65b) 6-chloro-3-nitropyridin-2-amine

To a stirred solution of compound 65a (12 g) in MeOH (50 mL) in a sealed tube was added 2M NH₃ in MeOH at 0° C. The reaction was gradually brought to ambient temperature and stirred for 16 h. Upon completion, the reaction was diluted with water and filtered under suction. The precipitate was dried to afford the required compound 65b (3.1 g)

### (65c) 6-methoxy-3-nitropyridin-2-amine

To an ice cold solution of MeOH (102 mL) under Nitrogen atmosphere was added Sodium (1.9 g) metal. After the complete solubilization of the Sodium, compound-65b (6.0 g) was added. The reaction was heated at 90° C. for 2 h. Upon completion, the solvent in the reaction was distilled out. The crude was partitioned between water and EtOAc. The EtOAc layers were dried over Na₂SO₄ and concentrated under reduced pressure to afford the required compound-65c (6.5 g).

### (65d) 6-methoxypyridine-2,3-diamine

To a stirred solution of compound-65c (4.5 g) in EtOH (80 mL) was added 10% Pd—C (2.13 g) under stirred under 1 atm hydrogen pressure for 16 h. Upon completion the reaction was diluted with EtOAc and filtered through celite bed. The filtrate was concentrated under reduced pressure to afford Compound-65d (3.65 g) as a black solid.

### (65e) 6-methoxypyrido[3,2-b]pyrazin-3(4H)-one

To a chilled solution of compound-65d (3.25 g) in MeOH (30 mL) was added ethylglyoxalate (50% in toluene) at 0° C. The reaction was gradually brought to ambient temperature and stirred for 16 h. Upon completion, the reaction was diluted with MeOH and filtered under suction. The obtained filtrate was concentrated. The resulting crude was purified by flash column chromatography eluting the required compound with 4% MeOH—CH₂Cl₂. Compound-65e (0.53 g) was thus obtained.

### (65f) Tert-butyl-1-(2-(6-methoxy-3-oxopyrido[3,2-b]pyrazin-4(3H)-yl)ethyl)piperidin-4-yl-carbamate

To a stirred solution of compound-65e (0.53 g) in DMF (5 mL) at 0° C., was added NaH (0.23 g, 60% in oil). The reaction was stirred at this temperature for 1 h. Then compound 26e (1.15 g) synthetic methods for which, are provided in detail in the parent U.S. Ser. No. 14/434,870 dissolved in DMF (5 mL) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-65f with 6% MeOH—CH₂Cl₂ as a brown solid. (0.35 g).

### (65g) 4-(2-(4-aminopiperidin-1-yl)ethyl)-6-methoxypyrido[3,2-b]pyrazin-3(4H)-one

To a chilled solution of compound-65f (0.35 g, 0.868 mmol) in CH₂Cl₂, was added TFA (2.8 ml) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-65g (0.27 g crude).

### (65h) VT-03-00101

To a chilled solution of compound-65g (0.27 g, 0.88 mmol) and 3-Fluoro-4-methyl benzaldehyde (0.122 g, 0.88 mmol) in EtOH (5 mL) was added Titanium isopropoxide (0.25 ml). The reaction was stirred at ambient temperature for 16 h. Then NaBH4 (0.33 g) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00101 with 6% MeOH—CH₂Cl₂ as a pale brown solid (0.012 g).

¹HNMR (400 MHz, CDCl₃) δ 8.56 (S, 1H), 8.05 (d, 1H), 7.19-7.21 (m, 2H), 7.0-7.1 (m, 3H), 4.60 (t, 2H), 4.12 (s, 3H), 3.75 (s, 2H), 3.01 (d, 2H), 2.85 (t, 2H), 2.55-2.65 (m, 1H), 2.25 (s, 3H), 2.20 (t, 2H), 1.90 (d, 3H), 1.48 (q, 2H), Mass spectra [M+H]+ m/z 426.00.

### (66) Synthesis of VT-03-00102

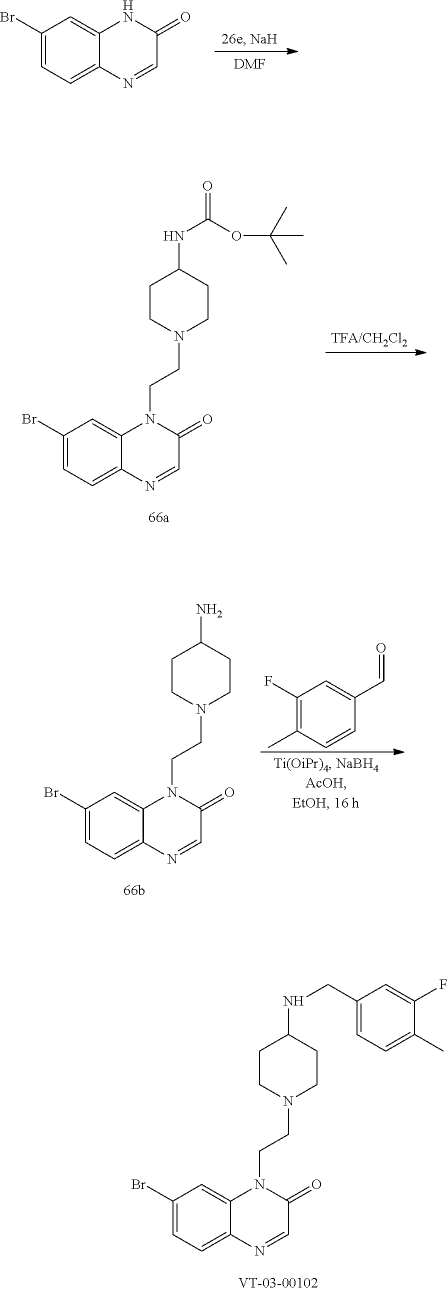

### (66a) Tert-butyl 1-(2-(7-bromo-2-oxoquinoxalin-1(2H)-yl)ethyl)piperidin-4-ylcarbamate

To a stirred solution of 7-bromoquinoxalin-2(1H)-one (0.4 g) in DMF (5 mL) at 0° C., was added NaH (0.085 g, 60% in oil). The reaction was stirred at this temperature for 1 h. Then compound 26e (0.86 g) dissolved in DMF (5 mL) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-66a with 4% MeOH—CH₂Cl₂ as a pale brown solid. (0.22 g).

### (66b) 1-(2-(4-aminopiperidin-1-yl)ethyl)-7-bromoquinoxalin-2(1H)-one

To a chilled solution of compound-66a (0.22 g, 0.488 mmol) in CH₂Cl₂ (10 mlL), was added TFA (1 ml) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-66b (0.11 g crude).

### (66c) VT-03-00102

To a chilled solution of compound-66b (0.11 g, 0.285 mmol) and 3-Fluoro-4-methyl benzaldehyde (0.047 g, 0.342 mmol) in EtOH (4 mL) was added Titanium isopropoxide (0.34 mL, 1.14 mmol). The reaction was stirred at ambient temperature for 16 h. Then NaBH₄ (0.021 g) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00102 with 8% MeOH—CH₂Cl₂ as a green viscous (0.020 g).

¹HNMR (400 MHz, CDCl₃): δ 8.22 (s, 1H), 7.72 (d, 1H), 7.65 (s, 1H), 7.45 (d, 2H), 7.12 (t, 1H), 7.0-7.1 (m, 2H), 4.32 (t, 2H), 3.79 (s, 2H), 2.97 (d, 2H), 2.65 (t, 2H), 2.56-2.66 (m, 1H), 2.25 (s, 3H), 2.20 (t, 2H), 1.86 (d, 2H), 1.46 (q, 2H) Mass spectra [M+H]+ m/z 475.2

### (67) Synthesis of VT-03-00103

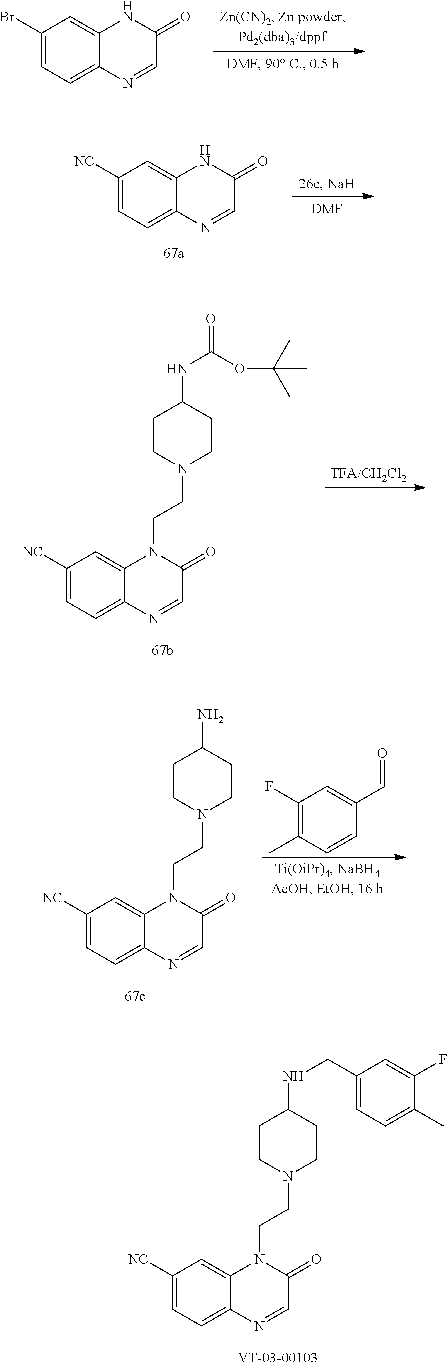

### (67a) 3-Oxo-3,4-dihydroquinoxaline-6-carbonitrile

A sealed tube was charged with (7-bromoquinoxalin-2(1H)-one (2.2 g, 9.82 mmol), Zn(CN)₂ (1.72 g, 14.73 mmol), Pd₂(dba)₃ (0.896 g, 0.98 mmol), dppf (0.542 mg, 0.98 mmol), Zn powder (0.126 g, 1.96 mmol) and DMF (20 mL). It was degassed for 10 min with Nitrogen and heated at 90° C. for 0.5 h. Upon completion, the reaction was diluted with EtOAc and filtered through a celite bed. The filtrate was concentrated. Silicagel (100-200 mesh) flash column chromatography of the crude eluted the required compound-67a with 50% EtOAc-Hexanes as a pale brown solid 67a (0.650 g).

### (67b) tert-butyl 1-(2-(7-cyano-2-oxoquinoxalin-1(2H)-yl)ethyl)piperidin-4-ylcarbamate

To a stirred solution of compound-67a (0.45 g) in DMF (7.5 ml) at 0° C., was added NaH (0.126 g, 60% in oil). The reaction was stirred at this temperature for 1 h. Then compound 26e (1 g) dissolved in DMF (7.5 ml) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-67b with 5% MeOH—CH₂Cl₂ as off white solid (0.32 g).

### (67c) 4-(2-(4-aminopiperidin-1-yl)ethyl)-3-oxo-3,4-dihydroquinoxaline-6-carbonitrile

To a chilled solution of compound-67b (0.3 g, 0.755 mmol) in CH₂Cl₂ (10 mL), was added TFA (1.5 mL) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-67c (0.15 g crude).

### (67d) VT-03-00103

To a chilled solution of compound-67c (0.1 g, 0.336 mmol) and 3-Fluoro-4-methyl benzaldehyde (0.055 g, 0.403 mmol) in EtOH (5 mL) was added Titanium isopropoxide (0.4 mL, 1.34 mmol). The reaction was stirred at ambient temperature for 16 h. Then NaBH₄ (0.025 g) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00103 with 8% MeOH—CH₂Cl₂ as a pale yellow solid (0.010 g).

¹HNMR (400 MHz, CDCl₃) δ 8.23 (s, 1H), 7.82 (d, 1H), 7.78 (s, 1H), 7.42 (d, 1H), 7.15 (t, 1H), 7.05 (t, 2H), 4.36 (t, 2H), 3.79 (s, 2H), 2.96 (d, 2H), 2.65 (t, 2H), 2.54-2.60 (m, 1H), 2.21 (s, 3H), 2.21 (t, 2H), 1.92 (d, 2H), 1.45 (q, 2H) Mass spectra [M+H]+ m/z 420.1

### (68) Synthesis of VT-03-00104

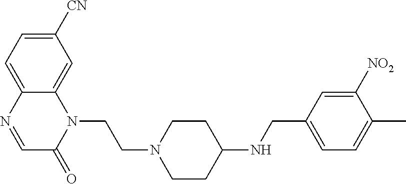 [SMILES: Cc1ccc(CNC2CCN(CCn3c(=O)cnc4ccc(C#N)cc43)CC2)cc1[N+](=O)[O-]]

To a chilled solution of compound-67c (0.1 g, 0.336 mmol) and 3-Nitro-4-methyl benzaldehyde (0.066 g, 0.403 mmol) in EtOH (5 mL) was added Titanium isopropoxide (0.4 mL, 1.34 mmol). The reaction was stirred at ambient temperature for 16 h. Then NaBH₄ (0.025 g) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00104 with 7% MeOH—CH₂Cl₂ as a pale brown solid (0.012 g).

¹HNMR (400 MHz, CDCl₃) δ 8.29 (s, 1H), 8.0 (s, 1H), 7.71 (d, 1H), 7.56 (d, 1H), 7.45 (d, 1H), 7.18 (d, 1H), 7.05 (d, 1H), 4.31 (t, 2H), 3.75 (s, 2H), 2.96 (d, 2H), 2.65 (t, 2H), 2.51-2.60 (m, 1H), 2.21 (s, 3H), 2.19 (t, 2H), 1.89 (d, 2H), 1.46 (q, 2H) Mass spectra [M+H]+ m/z 447.1

### (69) Synthesis of VT-03-00108

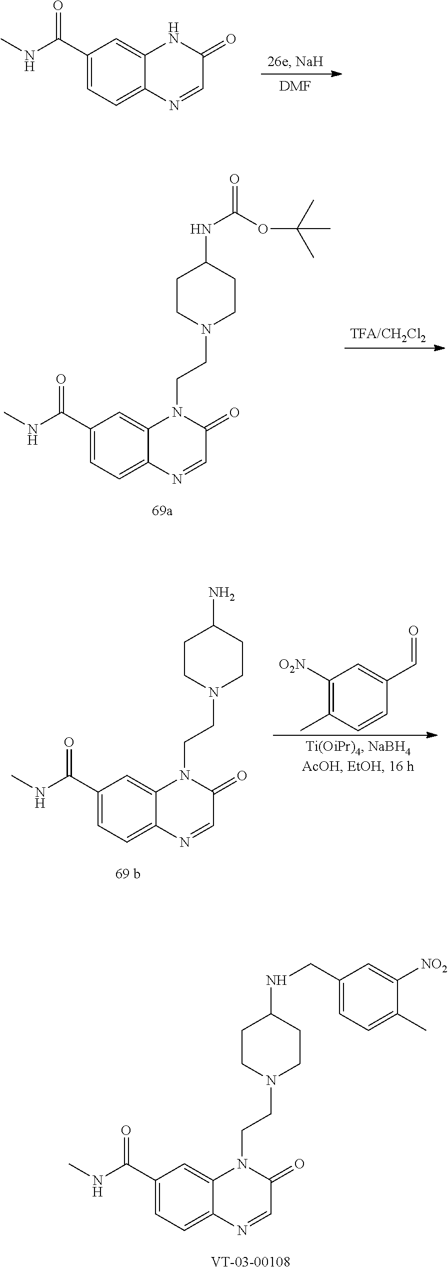

### (69a) Tert-butyl 1-(2-(7-(methylcarbamoyl)-2-oxoquinoxalin-1(2H)-yl)ethyl)piperidin-4-ylcarbamate

To a stirred solution of N-methyl-3-oxo-3,4-dihydroquinoxaline-6-carboxamide (0.8 g) in DMF (10 ml) at 0° C., was added NaH (0.190 g, 60% in oil). The reaction was stirred at this temperature for 1 h. Then compound 26e (1.52 g) dissolved in DMF (10 ml) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-69a with 4% MeOH—CH₂Cl₂ as an Pale brown solid (0.44 g).

### (69b) 4-(2-(4-aminopiperidin-1-yl)ethyl)-N-methyl-3-oxo-3,4-dihydroquinoxaline-6-carboxamide

To a chilled solution of compound-69a (0.21 g, 0.489 mmol) in CH₂Cl₂ (10 mL), was added TFA (1.2 mL) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-69b (0.15 g crude).

### (69c) VT-03-00108

To a chilled solution of compound-69b (0.15 g, 0.455 mmol) and 3-Nitro-4-methyl benzaldehyde (0.091 g, 0.546 mmol) in EtOH (4 mL) was added Titanium isopropoxide (0.5 ml, 1.82 mmol). The reaction was stirred at ambient temperature for 16 h. Then NaBH₄ (0.033 g) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00108 with 2% MeOH—CH₂Cl₂ as a green viscous (0.015 g).

¹HNMR (400 MHz, CDCl₃) δ 8.36 (s, 1H), 7.92 (d, 2H), 7.80-7.89 (m, 1H), 7.52 (d, 1H), 7.30-7.42 (m, 1H), 4.52 (t, 2H), 3.81 (s, 2H), 3.51 (s, 1H), 3.18-3.24 (m, 2H), 3.05 (s, 3H), 2.81-2.91 (m, 2H), 2.62 (s, 3H), 2.41-2.50 (m, 2H), 2.11-2.21 (m, 3H), 1.57-1.67 (m, 2H), Mass spectra [M+H]+ m/z 479.1

### (70) Synthesis of VT-03-00109

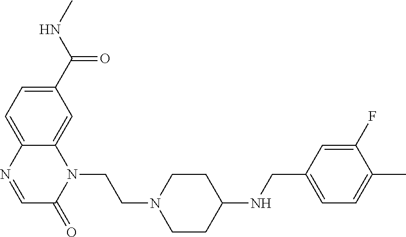 [SMILES: CNC(=O)c1ccc2ncc(=O)n(CCN3CCC(NCc4ccc(C)c(F)c4)CC3)c2c1]

To a chilled solution of compound-69b (0.15 g, 0.455 mmol) and 3-Fluoro-4-methyl benzaldehyde (0.075 g, 0.546 mmol) in EtOH (4 mL) was added Titanium isopropoxide (0.5 mL, 1.82 mmol). The reaction was stirred at ambient temperature for 16 h. Then NaBH₄ (0.033 g) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00109 with 2% MeOH—CH₂Cl₂ as a green viscous (0.019 g).

¹HNMR (400 MHz, CDCl₃) δ 8.36 (s, 1H), 8.10 (s, 1H), 7.92 (d, 1H), 7.75 (d, 1H), 7.16 (t, 1H), 6.92-6.98 (m, 3H), 4.46 (t, 2H), 3.76 (s, 2H), 3.09 (s, 6H), 2.79 (t, 2H), 2.61-2.71 (m, 1H), 2.22 (s, 3H), 1.96 (d, 3H), 1.54 (d, 2H), 1.36 (s, 2H) Mass spectra [M+H]+ m/z 452.1

### (71) Synthesis of VT-03-00110

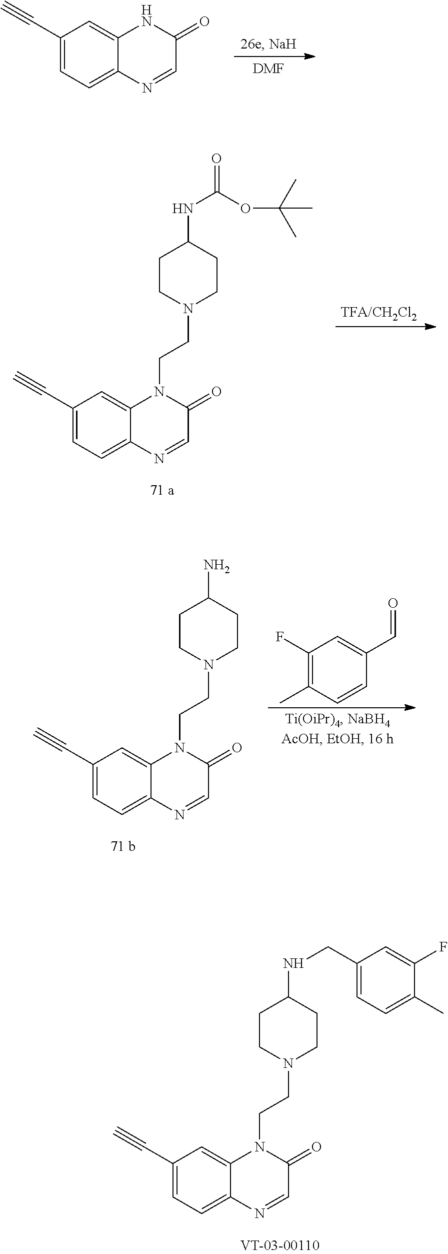

### (71a) Tert-butyl 1-(2-(7-ethynyl-2-oxoquinoxalin-1(2H)-yl)ethyl)piperidin-4-ylcarbamate

To a stirred solution of 7-ethynylquinoxalin-2(1H)-one (0.2 g) in DMF (4 ml) at 0° C., was added NaH (0.056 g, 60% in oil). The reaction was stirred at this temperature for 1 h. Then compound 26e (0.451 g) dissolved in DMF (4 ml) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-71a with 2% MeOH—CH₂Cl₂ as an Pale yellow viscous (0.13 g).

### (71b) 1-(2-(4-aminopiperidin-1-yl)ethyl)-7-ethynylquinoxalin-2(1H)-one

To a chilled solution of compound-71a (0.14 g, 0.35 mmol) in CH₂Cl₂ (10 mL), was added TFA (0.6 mL) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-71b (0.11 g crude).

### (71c) VT-03-00110

To a chilled solution of compound-71b (0.1 g, 0.337 mmol) and 3-Fluoro-4-methyl benzaldehyde (0.055 g, 0.404 mmol) in EtOH (5 mL) was added Titanium isopropoxide (0.4 mL, 1.34 mmol). The reaction was stirred at ambient temperature for 16 h. Then NaBH₄ (0.025 g) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00110 with 8% MeOH—CH₂Cl₂ as a green viscous (0.012 g).

¹HNMR (400 MHz, CDCl₃) δ 8.45 (s, 1H), 7.96 (d, 2H), 7.61 (d, 2H), 7.15 (t, 1H), 7.05-7.10 (m, 2H), 4.65 (t, 2H), 3.50 (s, 1H), 3.15 (d, 2H), 2.96 (t, 2H), 2.75-2.83 (m, 1H), 2.36-2.46 (m, 2H), 2.21 (s, 3H), 2.15-2.20 (m, 2H) 1.98-2.1 (m, 2H), 1.67 (q, 2H), 1.41 (s, 2H). Mass spectra [M+H]+ m/z 419.1

### (72) Synthesis of VT-03-00111

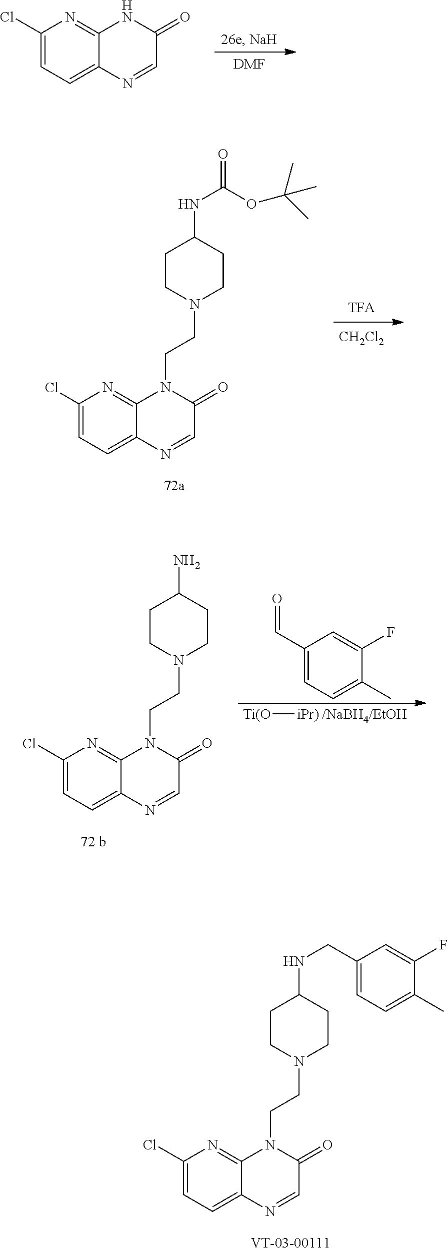

### (72a) Tert-butyl 1-(2-(6-chloro-3-oxopyrido[3,2-b]pyrazin-4(3H)-yl)ethyl)piperidin-4-ylcarbamate

To a stirred solution of 6-chloropyrido[3,2-b]pyrazin-3(4H)-one (0.2 g, 1.11 mmol) in DMF (5 mL) at 0° C., was added NaH (0.043 g, 60% in oil). The reaction was stirred at this temperature for 1 h. Then compound 26e (0.43 g) dissolved in DMF (5 mL) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by silica gel (100-200 mesh) Flash column chromatography eluting the required compound-72a with 70% EtOAc-Hexanes as a golden yellow viscous (0.31 g).

### (72b) 4-(2-(4-aminopiperidin-1-yl)ethyl)-6-chloropyrido[3,2-b]pyrazin-3(4H)-one

To a chilled solution of compound-72a (0.2 g) in CH₂Cl₂ (2.8 mL), was added TFA (1.2 mL) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-72b (0.103 g crude).

### (72c) VT-03-00111

To a chilled solution of compound-72b (0.1 g, 0.334 mmol) and 3-Fluoro-4-methyl benzaldehyde (0.046 g, 0.335 mmol) in EtOH (4 mL) was added Titanium isopropoxide (0.4 ml). The reaction was stirred at ambient temperature for 16 h. Then NaBH₄ (0.019 g) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00111 with 10% MeOH—CH₂Cl₂ as a green viscous (0.009 g).

¹HNMR (400 MHz, CDCl₃) δ 8.30 (s, 1H), 8.11 (d, 1H), 7.29 (s, 1H), 7.15 (t, 1H), 6.96-7.1 (m, 2H), 4.56 (t, 2H), 3.78 (s, 2H), 3.05 (d, 2H), 2.76 (t, 2H), 2.51-2.61 (m, 1H), 2.26 (s, 3H), 2.15 (t, 2H), 1.83 (d, 2H), 1.25 (d, 3H). Mass spectra [M+H]+ m/z 430.1

### (73) Synthesis of VT-03-00112

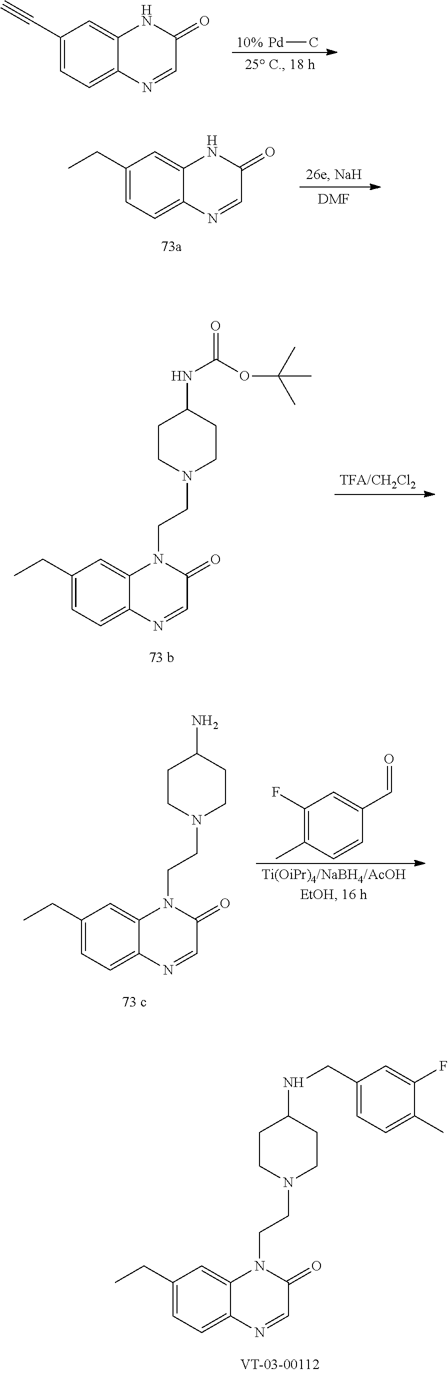

### (73a) 7-ethylquinoxalin-2(1H)-one

To a stirred solution of 7-ethynylquinoxalin-2(1H)-one (0.460 g, 2.70 mmol) in MeOH (20 mL) was added 10% Pd—C (0.3 g). The reaction was stirred under 1 atm Hydrogen pressure for 18 h. Upon completion, the reaction was passed through a celite bed and the filtrate was concentrated. The crude was triturated with n-Pentane to afford the required compound-73a as brown solid (0.350 g).

### (73b) Tert-butyl 1-(2-(7-ethyl-2-oxoquinoxalin-1(2H)-yl)ethyl)piperidin-4-ylcarbamate

To a stirred solution of compound-73a (0.35 g, 2.01 mmol) in DMF (5 mL) at 0° C., was added NaH (0.096 g, 60% in oil, 4.02 mmol). The reaction was stirred at this temperature for 1 h. Then compound 26e (0.776 g, 2.41 mmol) dissolved in DMF (5 ml) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) flash column chromatography eluting the required compound-73b with 80% EtOAc-Hexanes as yellow semisolid (0.18 g).

### (73c) 1-(2-(4-aminopiperidin-1-yl)ethyl)-7-ethylquinoxalin-2(1H)-one

To a chilled solution of compound-73b (0.180 g) in CH₂Cl₂ (8 mL), was added TFA (1 mL) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-73c (0.120 g crude).

### (73d) VT-03-00112

To a chilled solution of compound-73c (0.12 g, 0.4 mmol) and 3-Fluoro-4-methyl benzaldehyde (0.066 g, 0.48 mmol) in EtOH (4 mL) was added Titanium isopropoxide (0.5 mLl, 1.6 mmol). The reaction was stirred at ambient temperature for 16 h. Then NaBH₄ (0.030 g, 0.8 mmol) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00112 with 8% MeOH—CH₂Cl₂ as a green liquid (0.015 g).

¹HNMR (400 MHz, CDCl₃) δ 8.22 (s, 1H), 7.80 (d, 1H), 7.23-7.33 (m, 2H), 7.18 (t, 1H), 7.01 (t, 2H), 4.40 (t, 2H), 3.80 (s, 2H), 3.01 (d, 2H), 2.82 (q, 2H), 2.70 (t, 2H), 2.56-2.66 (m, 1H), 2.32 (s, 3H), 2.20 (t, 2H), 1.91 (d, 2H), 1.52 (q, 2H), 1.32 (t, 3H). Mass spectra [M+H]+ m/z 423.25

### (74) Synthesis of VT-03-00113

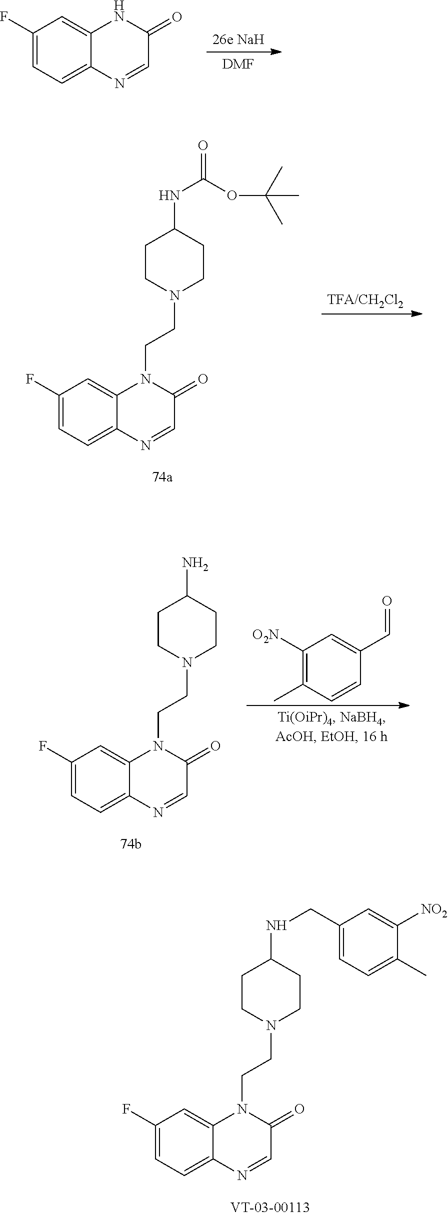

### (74a) tert-butyl 1-(2-(7-fluoro-2-oxoquinoxalin-1(2H)-yl)ethyl)piperidin-4-ylcarbamate

To a stirred solution of 7-fluoroquinoxalin-2(1H)-one (0.5 g, 3.048 mmol) in DMF (7.5 mL) at 0° C., was added NaH (0.146 g, 60% in oil, 6.1 mmol). The reaction was stirred at this temperature for 1 h. Then compound 26e (1.17 g, 3.658 mmol) dissolved in DMF (7.5 mL) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-74a with 80% EtOAc-Hexanes as a semisolid (0.35 g).

### (74b) 1-(2-(4-aminopiperidin-1-yl)ethyl)-7-fluoroquinoxalin-2(1H)-one

To a chilled solution of compound-74a (0.35 g) in CH₂Cl₂ (9 mL), was added TFA (1.6 mL) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-74b (0.240 g crude).

### (74c) VT-03-00113

To a chilled solution of compound-74b (0.15 g, 0.515 mmol) and 3-Nitro-4-methyl benzaldehyde (0.102 g, 0.618 mmol) in EtOH (5 mL) was added Titanium isopropoxide (0.61 mL, 2.06 mmol). The reaction was stirred at ambient temperature for 16 h. Then NaBH₄ (0.038 g, 1.03 mmol) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00113 with 2% MeOH—CH₂Cl₂ as a green liquid (0.030 g).

¹HNMR (400 MHz, CDCl₃) δ 8.21 (s, 1H), 7.91 (s, 1H), 7.82 (q, 1H), 7.51 (d, 1H), 7.3 (q, 1H), 7.15-7.23 (m, 2H), 4.31 (t, 2H), 3.82 (s, 2H), 2.92 (d, 2H), 2.65 (t, 2H), 2.60 (s, 3H), 2.51-2.61 (m, 1H), 2.22 (t, 2H), 1.91 (d, 2H), 1.41 (q, 2H) Mass spectra [M+H]+ m/z 440.1

### (75) Synthesis of VT-03-00114

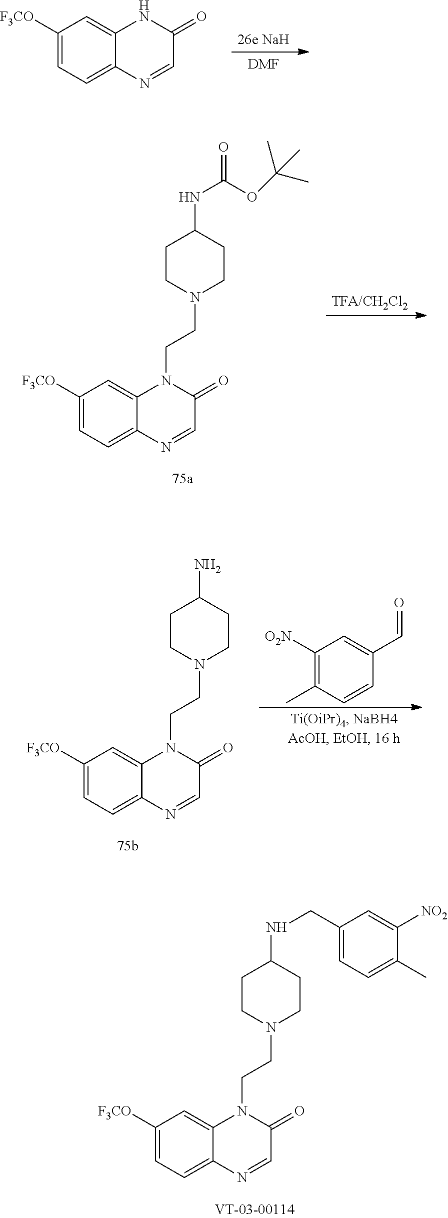

### (75a) Tert-butyl 1-(2-(2-oxo-7-(trifluoromethoxy)quinoxalin-1(2H)-yl)ethyl)piperidin-4-ylcarbamate

To a stirred solution of 7-(trifluoromethoxy)quinoxalin-2(1H)-one (0.5 g, 2.173 mmol) in DMF (7.5 mL) at 0° C., was added NaH (0.104 g, 60% in oil, 4.36 mmol). The reaction was stirred at this temperature for 1 h. Then compound 26e (0.840 g, 2.607 mmol) dissolved in DMF (7.5 mL) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-75a with 80% EtOAc-Hexanes as a golden yellow viscous (0.35 g).

### (75b) 1-(2-(4-aminopiperidin-1-yl)ethyl)-7-(trifluoromethoxy)quinoxalin-2(1H)-one

To a chilled solution of compound-75a (0.35 g, 0.767 mmol) in CH₂Cl₂ (9 ml), was added TFA (1.8 mL) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-75b (0.220 g crude).

### (75c) VT-03-00114

To a chilled solution of compound-75b (0.15 g, 0.421 mmol) and 3-Nitro-4-methyl benzaldehyde (0.083 g, 0.505 mmol) in EtOH (5 mL) was added Titanium isopropoxide (0.5 mL, 1.684 mmol). The reaction was stirred at ambient temperature for 16 h. Then NaBH₄ (0.031 g, 0.842 mmol) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00114 with 2% MeOH—CH₂Cl₂ as a green viscous (0.030 g).

¹HNMR (400 MHz, CDCl₃) δ 8.92 (s, 1H), 7.92 (s, 1H), 7.51 (d, 1H), 7.29 (d, 2H), 7.20 (d, 1H), 4.30 (t, 2H), 3.85 (s, 2H), 2.95 (d, 2H), 2.65 (t, 2H), 2.60 (s, 3H), 2.50-2.56 (m, 1H), 2.20 (t, 2H), 1.95 (d, 2H), 1.45 (q, 2H) Mass spectra [M+H]+ m/z 506.1

### (76) Synthesis of VT-03-00116

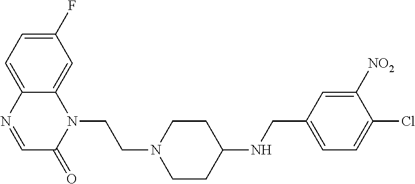 [SMILES: O=c1cnc2ccc(F)cc2n1CCN1CCC(NCc2ccc(Cl)c([N+](=O)[O-])c2)CC1]

To a stirred solution of compound-74b (0.422 g, 1.45 mmol) and 4-Chloro-3-nitrobenzaldehyde (0.27 g, 1.45 mmol) in CHCl₃ (14 ml) was added Sodium Triacetoxyborohydride (0.368 g, 1.74 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00116 with 6% MeOH—CH₂Cl₂ as a pale brown viscous (0.132 g).

¹HNMR (400 MHz, CDCl₃) δ 8.19 (s, 1H), 8.01 (s, 1H), 7.85 (t, 1H), 7.65 (t, 2H), 7.55 (d, 1H), 7.21 (t, 1H), 4.22 (t, 2H), 3.76 (s, 2H), 2.91 (d, 2H), 2.52 (t, 2H), 2.31 (s, 1H), 2.01 (t, 2H), 1.75 (d, 2H), 1.21 (d, 3H); Mass spectra [M+H]+ m/z 460.1

### (77) Synthesis of VT-03-00117

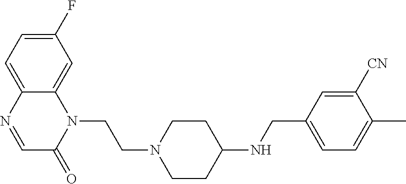 [SMILES: Cc1ccc(CNC2CCN(CCn3c(=O)cnc4ccc(F)cc43)CC2)cc1C#N]

To a stirred solution of compound-74b (0.6 g, 2.06 mmol) and 5-Formyl-2-methylbenzonitrile (0.3 g, 2.06 mmol) in CHCl₃ (16 mL) was added Sodium Triacetoxyborohydride (0.52 g, 2.47 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00117 with 6% MeOH—CH₂Cl₂ as a pale brown viscous (0.167 g).

¹HNMR (400 MHz, DMSO-d6) δ 8.19 (s, 1H), 7.85-7.95 (m, 1H), 7.65 (s, 1H), 7.54-7.60 (m, 2H), 7.38 (d, 1H), 7.21 (t, 1H), 4.22 (t, 2H), 3.75 (s, 2H), 2.85 (d, 2H), 2.57 (t, 2H), 2.41 (s, 3H), 2.30-2.36 (m, 1H), 2.0 (s, 2H), 1.78 (d, 2H), 1.20 (m, 2H), Mass spectra [M+H]+ m/z 420.1

### (78) Synthesis of VT-03-00118

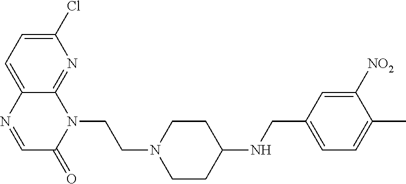 [SMILES: Cc1ccc(CNC2CCN(CCn3c(=O)cnc4ccc(Cl)nc43)CC2)cc1[N+](=O)[O-]]

To a stirred solution of compound-72b (0.45 g, 1.46 mmol) and 4-methyl-3-nitrobenzaldehyde (0.214 g, 1.461 mmol) in 1,2-Dichloroethane (5 mL) was added Sodium Triacetoxyborohydride (0.401 g, 1.89 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00118 with 11% MeOH—CH₂Cl₂ as a pale brown viscous (0.142 g).

¹HNMR (400 MHz, DMSO-d6) δ 8.31 (t, 2H), 7.91 (s, 1H), 7.58 (d, 1H), 7.52 (d, 1H), 7.40 (d, 1H), 4.34 (t, 2H), 3.78 (s, 2H), 2.85 (d, 2H), 2.56 (t, 2H), 2.42 (s, 3H), 2.28-2.35 (m, 1H), 1.98 (t, 2H), 1.75 (d, 2H), 1.5 (q, 2H), Mass spectra [M+H]+ m/z 457.00.

### (79) Synthesis of VT-03-00119

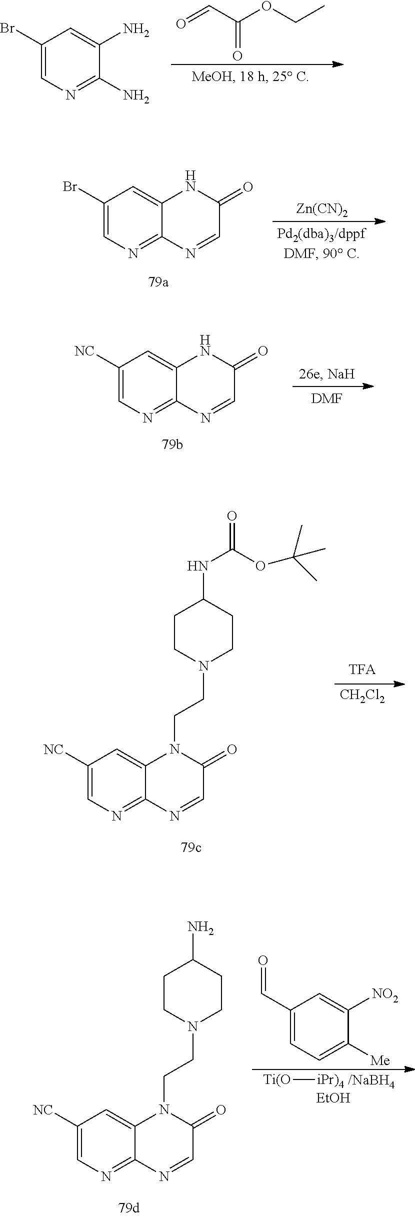

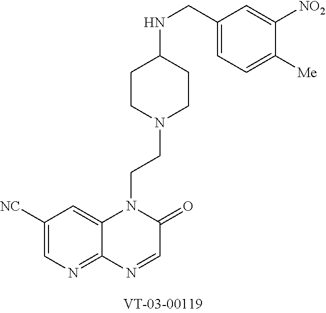 [SMILES: Cc1ccc(CNC2CCN(CCn3c(=O)cnc4ncc(C#N)cc43)CC2)cc1[N+](=O)[O-]]

### (79a) 7-bromopyrido[2,3-b]pyrazin-2(1H)-one

To a stirred solution of 2,3-Diamino-5-bromopyridine (5 g, 26.8 mmol) in MeOH (70 ml) was added Ethyl glyoxalate (50% in toluene, 25 ml) dropwise and stirred at ambient temperature for 18 h. Upon completion, the reaction mass was filtered through a celite bed and the filtrate was concentrated. Silicagel (100-200 mesh) flash column chromatography of the crude eluted the required compound-79a with 50% EtOAc-Hexanes as a pale brown solid (2.2 g).

### (79b) 2-oxo-1,2-dihydropyrido[2,3-b]pyrazine-7-carbonitrile

A sealed tube was charged with compound-79a (2.0 g, 8.88 mmol), Zn(CN)₂ (1.55 g, 13.32 mmol), Pd₂(dba)₃ (0.805 g, 0.88 mmol), dppf (0.476 g, 0.86 mmol), Zn powder (0.115 g, 1.77 mmol) and DMF (16 ml) was added. The reaction was heated at 90° C. for 30 min. Upon completion, the reaction was filtered through celite bed and the filtrate concentrated to dryness. Silicagel (100-200 mesh) flash column chromatography of the crude eluted the required compound-79b with 50% EtOAc-Hexanes as a dark brown solid (0.65 g).

### (79c) tert-butyl 1-(2-(7-cyano-2-oxopyrido[2,3-b]pyrazin-1(2H)-yl)ethyl)piperidin-4-ylcarbamate

To a stirred solution of compound-79b (0.55 g, 3.197 mmol) in DMF (7.5 mL) at 0° C., was added NaH (0.153 g, 60% in oil, 6.394 mmol). The reaction was stirred at this temperature for 1 h. Then compound 26e (1.23 g, 3.836 mmol) dissolved in DMF (7.5 mL) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-79c with 100% EtOAc as a golden yellow viscous (0.31 g).

### (79d) 1-(2-(4-aminopiperidin-1-yl)ethyl)-2-oxo-1,2-dihydropyrido[2,3-b]pyrazine-7-carbonitrile

To a chilled solution of compound-79c (0.3 g, 0.75 mmol) in CH₂Cl₂ (10 mL), was added TFA (2 mL) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-79d (0.140 g crude).

### (79e) VT-03-00119

To a chilled solution of compound-79d (0.1 g, 0.336 mmol) and 3-Nitro-4-methyl benzaldehyde (0.066 g, 0.403 mmol) in EtOH (5 mL) was added Titanium isopropoxide (0.4 mL, 1.344 mmol). The reaction was stirred at ambient temperature for 16 h. Then NaBH₄ (0.025 g, 0.672 mmol) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00119 with 6% MeOH—CH₂Cl₂ as a yellow viscous (0.007 g).

¹HNMR (400 MHz, CDCl₃) δ 8.22 (s, 1H), 7.92 (s, 1H), 7.91 (d, 1H), 7.50 (d, 1H), 7.25 (d, 1H), 7.20 (d, 1H), 4.30 (t, 2H), 3.82 (s, 2H), 2.92 (d, 2H), 2.76 (t, 2H), 2.61 (s, 3H), 2.31-2.40 (m, 1H), 2.20 (t, 2H), 1.92 (d, 2H), 1.56 (t, 2H) Mass spectra [M+H]+ m/z 448.2

### (80) Synthesis of VT-03-00120

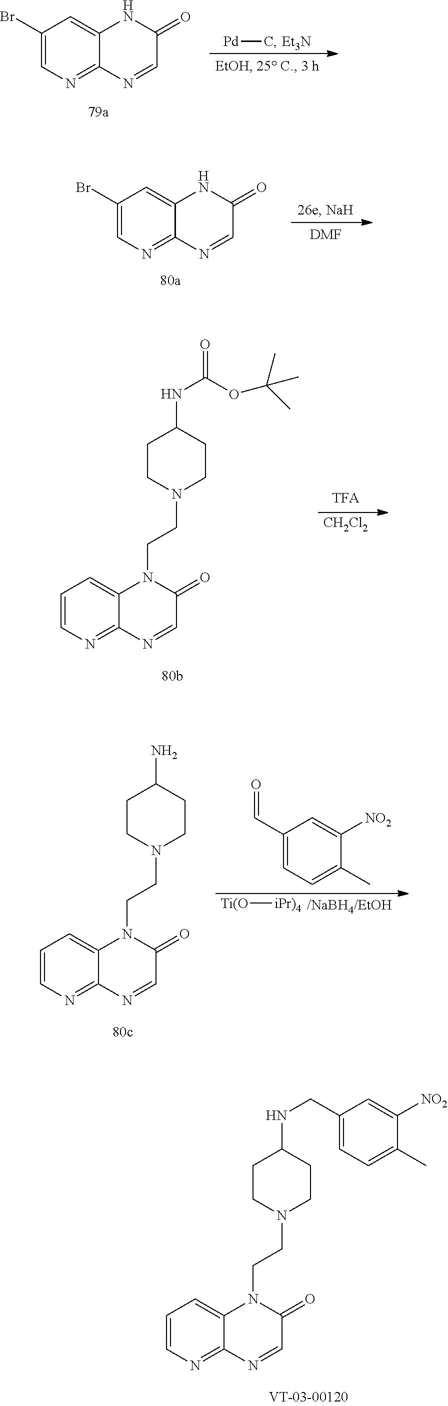

### (80a) Pyrido[2,3-b]pyrazin-2(1H)-one

To a stirred solution of 7-bromopyrido[2,3-b]pyrazin-2(1H)-one (2.2 g, 9.77 mmol) in EtOH (30 mL) was added Et₃N (2 mL) followed by 10% Pd—C (1 g). The reaction was stirred under 1 atm Hydrogen pressure for 16 h. The reaction mass was filtered through a celite bed and concentrated under reduced pressure. The obtained crude was purified by Silica gel (100-200 mesh) Flash column chromatography eluting the required compound-80a with 90% EtOAc in Hexanes as a brown solid (0.725 g).

### (80b) tert-butyl 1-(2-(2-oxopyrido[2,3-b]pyrazin-1(2H)-yl)ethyl)piperidin-4-ylcarbamate

To a stirred solution of compound-80a (0.4 g, 2.74 mmol) in DMF (7.5 mL) at 0° C., was added NaH (0.131 g, 60% in oil, 5.48 mmol). The reaction was stirred at this temperature for 1 h. Then compound 26e (1.05 g, 3.28 mmol) dissolved in DMF (7.5 mL) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-80b with 100% EtOAc as a golden yellow viscous (0.3 g).

### (80c) 1-(2-(4-aminopiperidin-1-yl)ethyl)-2-oxo-1,2-dihydropyrido[2,3-b]pyrazine-7-carbonitrile

To a chilled solution of compound-80b (0.3 g, 0.804 mmol) in CH₂Cl₂ (10 mL), was added TFA (1.5 mLl) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-80c (0.150 g crude).

### (80d) VT-03-00120

To a chilled solution of compound-80c (0.150 g, 0.551 mmol) and 3-Nitro-4-methyl benzaldehyde (0.110 g, 0.661 mmol) in EtOH (10 mL) was added Titanium isopropoxide (0.65 ml, 2.204 mmol). The reaction was stirred at ambient temperature for 16 h. Then NaBH₄ (0.040 g, 1.102 mmol) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00120 with 4% MeOH—CH₂Cl₂ as a green viscous (0.008 g).

¹HNMR (400 MHz, CDCl₃) δ 8.21 (s, 1H), 7.92 (s, 1H), 7.82 (q, 1H), 7.51 (d, 1H), 7.23 (d, 1H), 7.11-7.20 (m, 2H), 4.31 (t, 2H), 3.83 (s, 2H), 2.92 (d, 2H), 2.65 (t, 2H), 2.58 (s, 3H), 2.51-2.55 (m, 1H), 2.20 (t, 2H), 1.91 (d, 2H), 1.42 (q, 2H) Mass spectra [M+H]+ m/z 423.2

### (81) Synthesis of VT-03-00121

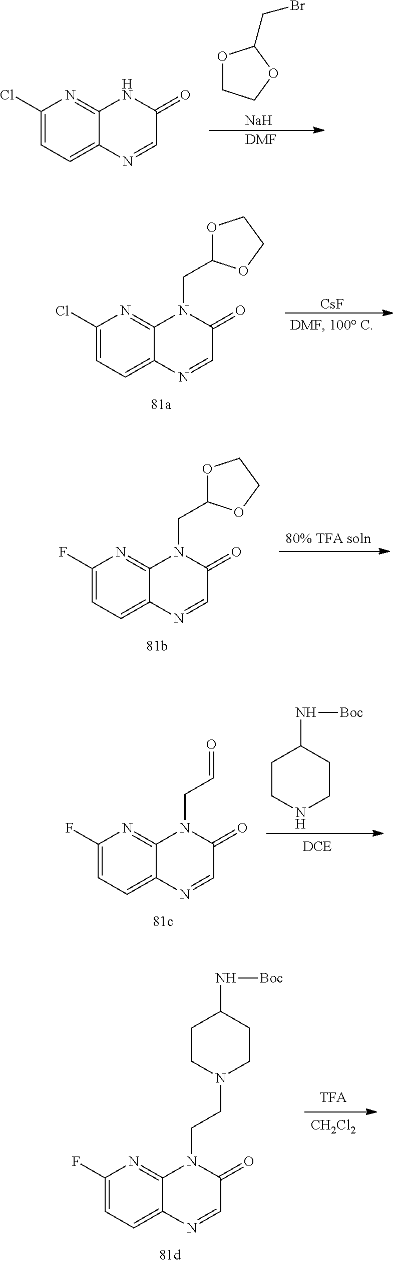

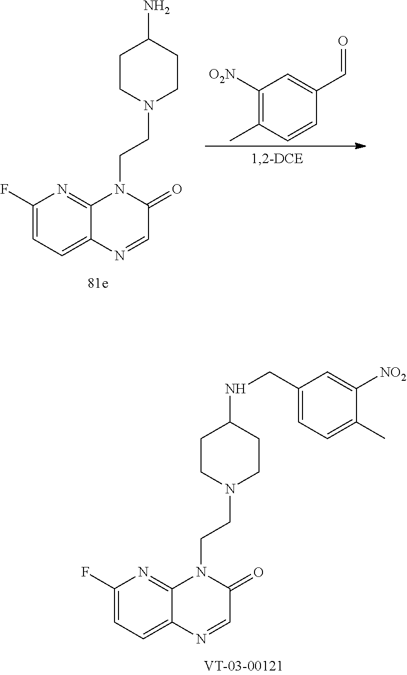 [SMILES: NC1CCN(CCn2c(=O)cnc3ccc(F)nc32)CC1 ; Cc1ccc(C=O)cc1[N+](=O)[O-] ; Cc1ccc(CNC2CCN(CCn3c(=O)cnc4ccc(F)nc43)CC2)cc1[N+](=O)[O-]]

### (81a) 4-((1,3-dioxolan-2-yl)methyl)-6-chloropyrido[3,2-b]pyrazin-3(4H)-one

To a stirred solution of 6-chloropyrido[3,2-b]pyrazin-3(4H)-one (4.0 g, 22.01 mmol) in DMF (50 mL) was added NaH (1.32 g) and heated at 50° C. for 40 min. Then 2-Bromomethyl-1,3-dioxolane (6.8 ml, 66.1 ml) was added dropwise and heated at 100° C. for 2 h. Upon completion the DMF in the reaction was distilled out. The crude was partitioned between water and EtOAc. The organic layers were dried over Na₂SO₄ and concentrated. Silicagel (100-200 mesh) flash column chromatography of the crude eluted the required compound-81a with 30% EtOAc-Hexanes as a brown solid (0.613 g).

### (81b) 4-((1,3-dioxolan-2-yl)methyl)-6-fluoropyrido[3,2-b]pyrazin-3(4H)-one

To a stirred solution of compound-81a (0.613 g, 2.28 mmol) in DMF (42 mL) was added CsF (3.46 g, 22.8 mmol) and heated at 100° C. for 1 h. Upon completion, the solvent in the reaction was distilled out. The crude was partitioned between water and EtOAc. The organic layers were dried over Na₂SO₄ and concentrated. Silicagel (100-200 mesh) flash column chromatography of the crude eluted the required compound-81b with 40% EtOAc-Petether as a brown solid (0.380 g).

### (81c) 2-(6-fluoro-3-oxopyrido[3,2-b]pyrazin-4(3H)-yl)acetaldehyde

A stirred solution of compound-81b (0.38 g) in 80% TFA (15 ml) was heated at 65° C. for 1 h. Upon completion, the solvent in the reaction was distilled out. The crude was basified (pH=8) with 1N NaOH solution and extracted with EtOAc. The organic layers were dried over Na₂SO₄ and concentrated under reduced pressure. Silicagel (100-200 mesh) flash column chromatography of the crude eluted the required compound-81c with 50% EtOAc-Hexanes as a brown solid (0.2 g).

### (81d) tert-butyl 1-(2-(6-fluoro-3-oxopyrido[3,2-b]pyrazin-4(3H)-yl)ethyl)piperidin-4-ylcarbamate

To a stirred solution of compound-81c (0.2 g, 0.964 mmol) and 4-(N-Boc-amino)piperidine (0.186 g, 0.964 mmol) in 1,2-DCE (12 mL) was added Sodium triacetoxyborohydride (0.244 g, 1.45 mmol) and stirred at ambient temperature for 4 h. Upon completion, the solvent in the reaction was distilled out. The crude was partitioned between water and 10% MeOH—CH₂Cl₂. The organic layers were dried over Na₂SO₄ and concentrated under reduced pressure to afford the required compound-81d as a green viscous (0.225 g crude).

### (81e) 4-(2-(4-aminopiperidin-1-yl)ethyl)-6-fluoropyrido[3,2-b]pyrazin-3(4H)-one

To a chilled solution of compound-81d (0.32 g) in CH₂Cl₂ (5 ml), was added TFA (1.5 ml) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-81e (0.220 g crude).

### (81f) VT-03-00121

To a stirred solution of compound-81e (0.16 g) and 4-methyl-3-nitrobenzaldehyde (0.09 g, 0.556 mmol) in 1,2-Dichloroethane (5 ml) was added Sodium Triacetoxyborohydride (0.140 g, 0.667 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00121 with 4% MeOH—CH₂Cl₂ as a pale brown solid (0.035 g).

¹HNMR (400 MHz, CDCl₃) δ 8.25-8.31 (m, 2H), 7.92 (s, 1H), 7.54 (d, 1H), 7.21 (s, 1H), 6.90 (d, 1H), 4.52 (t, 2H), 3.82 (s, 2H), 3.05 (d, 2H), 2.76 (t, 2H), 2.62 (s, 3H), 2.56-2.60 (m, 1H), 2.22-2.35 (m, 2H), 1.92 (t, 3H), 1.35 (q, 2H) Mass spectra [M+H]+ m/z 441.3

### (82) Synthesis of VT-03-00122

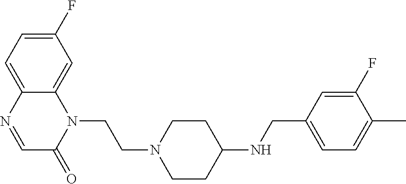 [SMILES: Cc1ccc(CNC2CCN(CCn3c(=O)cnc4ccc(F)cc43)CC2)cc1F]

To a stirred solution of compound-74b (0.21 g, 0.723 mmol) and 4-methyl-3-fluorobenzaldehyde (0.12 g, 0.723 mmol) in CHCl₃ (7 mL) was added Sodium Triacetoxyborohydride (0.18 g, 0.867 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00122 with 6% MeOH—CH₂Cl₂ as a pale brown solid (0.067 g).

¹HNMR (400 MHz, DMSO-d6) δ 8.19 (s, 1H), 7.82 (q, 1H), 7.50 (dd, 1H), 7.15-7.26 (m, 2H), 7.01-7.09 (m, 2H), 4.25-4.35 (m, 2H), 3.65 (s, 2H), 2.85 (d, 2H), 2.59 (t, 2H), 2.39-2.42 (m, 1H), 2.19 (s, 3H), 2.01 (t, 2H), 1.90 (s, 2H), 1.75 (d, 2H), 1.21-1.31 (m, 2H), Mass spectra [M+H]+ m/z 413.00.

### (83) Synthesis of VT-03-00124

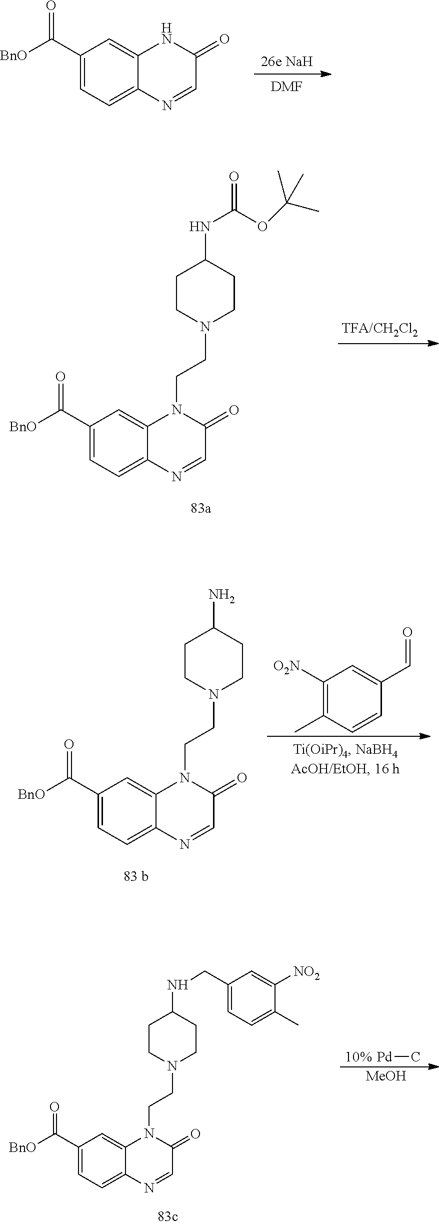

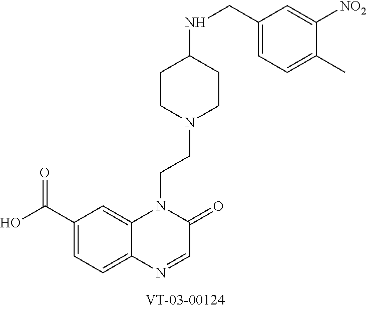 [SMILES: Cc1ccc(CNC2CCN(CCn3c(=O)cnc4ccc(C(=O)O)cc43)CC2)cc1[N+](=O)[O-]]

### (83a) benzyl 4-(2-(4-(tert-butoxycarbonylamino)piperidin-1-yl)ethyl)-3-oxo-3,4-dihydroquinoxaline-6-carboxylate

To a stirred solution of benzyl 3-oxo-3,4-dihydroquinoxaline-6-carboxylate (0.5 g, 1.78 mmol) in DMF (7.5 mlml) at 0° C., was added NaH (0.085 g, 60% in oil, 3.57 mmol). The reaction was stirred at this temperature for 1 h. Then compound 26e (0.690 g, 2.142 mmol) dissolved in DMF (7.5 ml) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-83a with 90% EtOAc-Hexanes as a brown viscous (0.29 g).

### (83b) benzyl 4-(2-(4-aminopiperidin-1-yl)ethyl)-3-oxo-3,4-dihydroquinoxaline-6-carboxylate

To a chilled solution of compound-83a (0.29 g, 0.573 mmol) in CH₂Cl₂ (10 mL), was added TFA (1.5 mL) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-83b (0.150 g crude).

### (83c) Benzyl-4-(2-(4-(4-methyl-3-nitrobenzylamino)piperidin-1-yl)ethyl)-3-oxo-3,4-dihydroquinoxaline-6-carboxylate

To a chilled solution of compound-83b (0.15 g, 0.369 mmol) and 3-Nitro-4-methyl benzaldehyde (0.073 g, 0.442 mmol) in EtOH (5 ml) was added Titanium isopropoxide (0.4 ml, 1.476 mmol). The reaction was stirred at ambient temperature for 16 h. Then NaBH₄ (0.028 g, 0.738 mmol) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound-83c with 5% MeOH—CH₂Cl₂ as a green viscous (0.030 g).

### (83d) VT-03-00124

Compound-83c (30 mg) was taken in MeOH (6 ml) and triturated with 10% Pd—C (50 mg) under Hydrogen atmosphere. Upon completion the reaction was filtered through celite bed and concentrated. The crude was triturated with EtOAc to give the required compound VT-03-00124 as a brown solid (7 mg).

¹HNMR (400 MHz, CDCl₃) δ 8.30 (s, 1H), 7.92 (s, 1H), 7.89 (d, 1H), 7.51 (d, 1H), 7.32 (s, 2H), 7.20 (d, 1H), 4.32 (t, 2H), 3.90 (s, 2H), 2.95 (d, 2H), 2.80 (s, 3H), 2.72 (t, 2H), 2.56-2.61 (m, 2H), 2.21 (t, 2H), 1.91 (d, 2H), 1.20-1.25 (m, 3H) Mass spectra [M+H]+ m/z 420.3

### (84) Synthesis of VT-03-00125

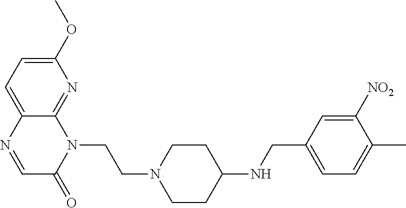 [SMILES: COc1ccc2ncc(=O)n(CCN3CCC(NCc4ccc(C)c([N+](=O)[O-])c4)CC3)c2n1]

To a chilled solution of compound-65g (0.42 g, 1.40 mmol) and 3-Nitro-4-methyl benzaldehyde (0.277 g, 1.68 mmol) in EtOH (10 ml) was added Titanium isopropoxide (0.6 ml, 2.1 mmol). The reaction was stirred at ambient temperature for 16 h. Then NaBH₄ (0.05 g, 1.54 mmol) followed by two drops of AcOH were added and stirred for 3 h. Upon completion, the solvent in the reaction was distilled out. The crude was partitioned between water and 10% MeOH—CH₂Cl₂. The organic layers were dried over Na₂SO₄ and concentrated under reduced pressure. Silicagel (100-200 mesh) flash column chromatography of the crude eluted the required compound VT-03-00125 with 8% MeOH—CH₂Cl₂ as a brown solid (0.121 g).

¹HNMR (400 MHz, CDCl₃) δ 8.55 (s, 1H), 8.05 (d, 1H), 7.93 (s, 1H), 7.51 (d, 1H), 7.25 (d, 1H), 7.15 (d, 1H), 4.55 (t, 2H), 4.15 (s, 3H), 3.85 (s, 2H), 3.01 (d, 2H), 2.61 (s, 3H), 2.55 (s, 1H), 2.20-2.26 (m, 2H), 1.95 (d, 3H), 1.27-1.37 (m, 2H), Mass spectra [M+H]+ m/z 453.3.

### (85) Synthesis of VT-03-00126

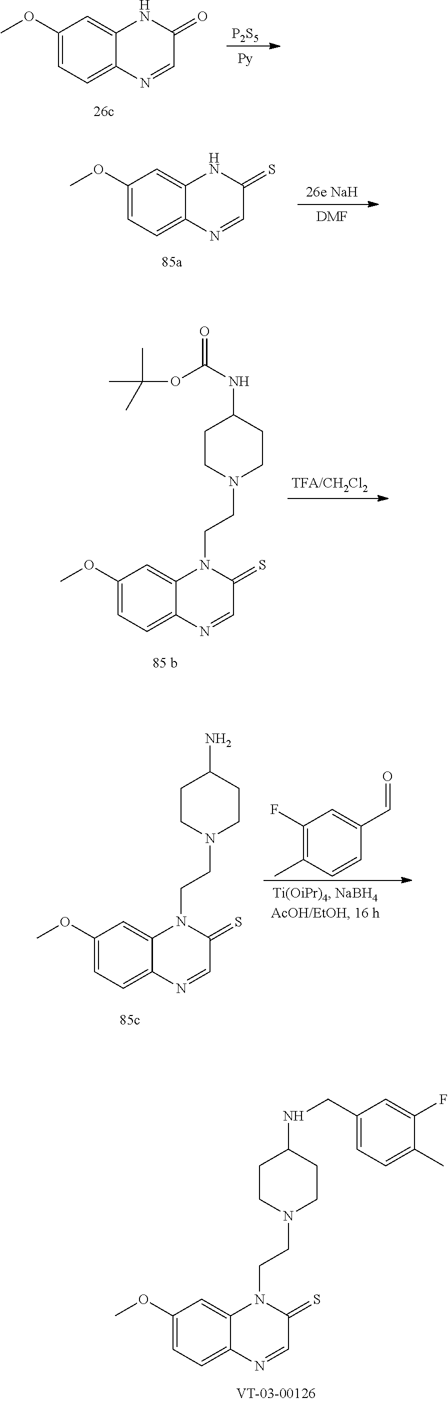

### (85a) 7-methoxyquinoxaline-2(1H)-thione

To a stirred solution of compound-26c (0.6 g, 3.40 mmol) in Pyridine (10 ml) was added P₂S₅ (2.7 g, 6.16 mmol) and heated at 130° C. for 6 h. Upon completion, the Pyridine in the reaction was distilled out. Silicagel (100-200 mesh) flash column chromatography of the crude eluted the required compound-85a with 11% EtOAc-Petether as a brown solid (0.65 g).

### (85b) tert-butyl 1-(2-(7-methoxy-2-thioxoquinoxalin-1(2H)-yl)ethyl)piperidin-4-ylcarbamate

To a stirred solution of compound-85a (0.45 g, 2.38 mmol) in DMF (13.5 mLl) at 0° C., was added NaH (0.104 g, 60% in oil, 2.6 mmol). The reaction was stirred at this temperature for 1 h. Then compound 26e (0.92 g, 2.86 mmol) dissolved in DMF (7.5 ml) was added dropwise at 0° C. The reaction was stirred at ambient temperature for 16 h. Upon completion the solvent in the reaction was distilled out. The crude was partitioned between water and 10% MeOH—CH₂Cl₂. The organic layers were dried over Na₂SO₄ and concentrated under reduced pressure. The obtained crude was purified by Silica gel (100-200 mesh) Flash column chromatography eluting the required compound-85b with 90% EtOAc-Hexanes as a brown viscous (0.44 g).

### (85c) 1-(2-(4-aminopiperidin-1-yl)ethyl)-7-methoxyquinoxaline-2(1H)-thione

To a chilled solution of compound-85b (0.44 g, 1.05 mmol) in CH₂Cl₂ (5 ml), was added TFA (0.97 ml) dropwise at 0° C. The reaction was stirred at ambient temperature for 2 h. Upon completion, the solvent in the reaction was distilled out. The crude was diluted with 10% MeOH—CH₂Cl₂ and washed with saturated NaHCO₃ solution. The organic layer was dried over Na₂SO₄ and concentrated under reduced pressure to afford the required compound-85c (0.210 g crude).

### (85d) VT-03-00126

To a stirred solution of compound-85c (0.1 g, 0.313 mmol) and 4-methyl-3-fluorobenzaldehyde (0.05 g, 0.313 mmol) in CHCl₃ (5 mL) was added Sodium Triacetoxyborohydride (0.079 g, 0.376 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00126 with 7% MeOH—CH₂Cl₂ as a dark green solid (0.010 g).

¹HNMR (400 MHz, CDCl₃) δ 8.41 (s, 1H), 7.90 (d, 1H), 7.21-7.31 (m, 5H), 3.96 (s, 3H), 3.91 (s, 2H), 3.51 (t, 2H), 3.21 (d, 2H), 2.85-2.95 (m, 3H), 2.75-2.80 (m, 2H), 2.21 (s, 3H), 1.9-2.0 (m, 2H), 1.25-1.31 (m, 3H), Mass spectra [M+H]+ m/z 441.2

### (86) Synthesis of VT-03-00127

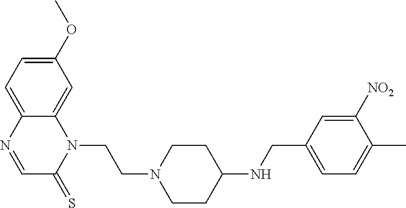 [SMILES: COc1ccc2ncc(=S)n(CCN3CCC(NCc4ccc(C)c([N+](=O)[O-])c4)CC3)c2c1]

To a stirred solution of compound-85c (0.21 g, 0.659 mmol) and 4-methyl-3-nitrobenzaldehyde (0.1 g, 0.659 mmol) in CHCl₃ (5 ml) was added Sodium Triacetoxyborohydride (0.16 g, 0.79 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00127 with 7% MeOH—CH₂Cl₂ as a pale brown viscous (0.015 g).

¹HNMR (400 MHz, CDCl₃) δ 8.41 (s, 1H), 7.92 (s, 1H), 7.82 (d, 1H), 7.50 (d, 1H), 7.31 (d, 1H), 7.22 (d, 2H), 3.96 (s, 3H), 3.85 (s, 2H), 3.54-3.64 (m, 2H), 3.15 (m, 2H), 2.85-2.95 (m, 2H), 2.58 (s, 3H), 3.35-3.45 (m, 2H), 2.05-3.15 (m, 2H), 0.91 (t, 2H) Mass spectra [M+H]+ m/z 468.1

### (87) Synthesis of VT-03-00128

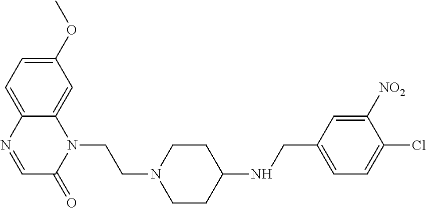 [SMILES: COc1ccc2ncc(=O)n(CCN3CCC(NCc4ccc(Cl)c([N+](=O)[O-])c4)CC3)c2c1]

To a stirred solution of compound-26g (0.4 g, 1.32 mmol) synthetic methods for which, are provided in detail in the parent U.S. application Ser. No. 14/434,870 and 4-Chloro-3-nitrobenzaldehyde (0.24 g, 1.32 mmol) in CHCl₃ (14 mL) was added Sodium Triacetoxyborohydride (0.325 g, 1.58 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00128 with 6% MeOH—CH₂Cl₂ as a yellow solid (0.063 g).

¹HNMR (400 MHz, DMSO-d6) δ 8.01 (s, 2H), 7.65-7.72 (m, 3H), 7.01 (s, 2H), 4.30 (t, 2H), 3.91 (s, 3H), 3.75 (s, 2H), 2.91 (d, 2H), 2.52 (t, 2H), 2.35-2.44 (m, 1H), 2.01 (t, 2H), 1.75 (d, 2H), 1.21 (t, 2H), Mass spectra [M+H]+ m/z 472.1

### (88) Synthesis of VT-03-00131

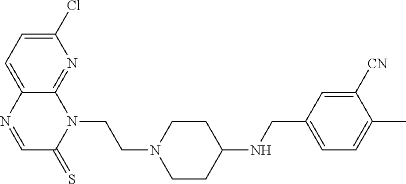 [SMILES: Cc1ccc(CNC2CCN(CCn3c(=S)cnc4ccc(Cl)nc43)CC2)cc1C#N]

To a stirred solution of compound-72b (0.2 g, 0.649 mmol) and 5-Formyl-2-methylbenzonitrile (0.094 g, 0.65 mmol) in CHCl₃ (6 mL) was added Sodium Triacetoxyborohydride (0.178 g, 1.3 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00131 with 6% MeOH—CH₂Cl₂ as a brown solid (0.059 g).

¹HNMR (400 MHz, DMSO-d6) δ 8.35 (t, 2H), 7.65 (s, 1H), 7.52 (t, 2H), 7.38 (d, 1H), 4.35 (t, 2H), 3.65 (s, 2H), 2.85 (d, 2H), 2.78 (t, 3H), 2.40 (s, 3H), 2.25-2.35 (m, 1H), 1.98-2.05 (m, 2H), 1.75 (d, 2H), 1.15 (q, 2H), Mass spectra [M+H]+ m/z 437.20.

### (89) Synthesis of VT-03-00132

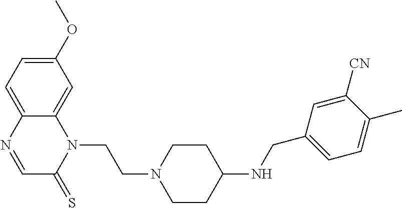 [SMILES: COc1ccc2ncc(=S)n(CCN3CCC(NCc4ccc(C)c(C#N)c4)CC3)c2c1]

To a stirred solution of compound-85c (0.21 g, 0.659 mmol) and 5-Formyl-2-methylbenzonitrile (0.094 g, 0.659 mmol) in CHCl₃ (10 mLl) was added Sodium Triacetoxyborohydride (0.16 g, 0.79 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00132 with 8% MeOH—CH₂Cl₂ as a brown viscous (0.017 g).

¹HNMR (400 MHz, CDCl₃) δ 8.41 (s, 1H), 7.90 (d, 1H), 7.21-7.33 (m, 5H), 3.96 (s, 3H), 3.91 (s, 2H), 3.51 (t, 2H), 3.21 (d, 2H), 2.85-2.90 (m, 3H), 2.75-2.80 (m, 2H), 2.21 (s, 3H), 1.9-2.05 (m, 2H), 1.25-1.35 (m, 3H), Mass spectra [M+H]+ m/z 448.2

### (90) Synthesis of VT-03-00133

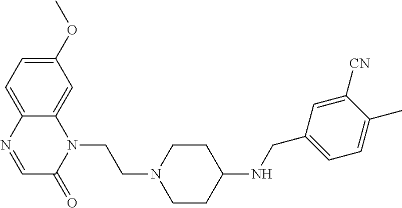 [SMILES: COc1ccc2ncc(=O)n(CCN3CCC(NCc4ccc(C)c(C#N)c4)CC3)c2c1]

To a stirred solution of compound-26g (0.3 g, 0.993 mmol) and 5-Formyl-2-methylbenzonitrile (0.158 g, 0.993 mmol) in CHCl₃ (10 mL) was added Sodium Triacetoxyborohydride (0.251 g, 1.19 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00133 with 6% MeOH—CH₂Cl₂ as a yellow solid (0.067 g).

¹HNMR (400 MHz, DMSO-d6) δ 8.01 (s, 1H), 7.75 (t, 2H), 7.45 (d, 1H), 7.38 (d, 1H), 6.98 (d, 2H), 4.30 (t, 2H), 3.91 (s, 3H), 3.70 (d, 2H), 2.90 (d, 2H), 2.52 (t, 2H), 2.41 (t, 2H), 2.35-2.39 (m, 1H), 2.0 (t, 2H), 1.75 (d, 2H), 1.18 (s, 4H), Mass spectra [M+H]+ m/z 432.30

### (91) Synthesis of VT-03-00134

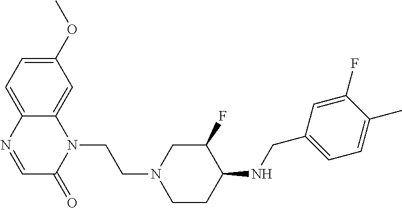 [SMILES: COc1ccc2ncc(=O)n(CCN3CC[C@H](NCc4ccc(C)c(F)c4)[C@H](F)C3)c2c1]

To a stirred solution of 1-(2-((3R,4S)-4-amino-3-fluoropiperidin-1-yl)ethyl)-7-methoxyquinoxalin-2(1H)-one (0.1 g, 0.312 mmol) and 4-methyl-3-nitrobenzaldehyde (0.057 g, 0.343 mmol) in CHCl₃ (5 ml) was added Sodium Triacetoxyborohydride (0.098 g, 0.468 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00134 with 7% MeOH—CH₂Cl₂ as a pale green solid (0.022 g).

¹HNMR (400 MHz, CDCl₃) δ 8.15 (s, 1H), 7.98 (s, 1H), 7.79 (d, 1H), 7.51 (d, 1H), 7.25 (d, 1H), 6.92 (d, 2H), 6.85 (s, 1H), 5.80 (d, 1H), 5.35 (m, 2H), 4.92 (s, 3H), 4.89 (s, 2H), 3.25-3.46 (m, 1H), 3.05 (d, 1H), 2.78-2.85 (m, 2H), 2.59 (s, 3H), 2.35-2.45 (m, 2H), 1.80 (m, 3H), Mass spectra [M+H]+ m/z 470.20.

### (92) Synthesis of VT-03-00135

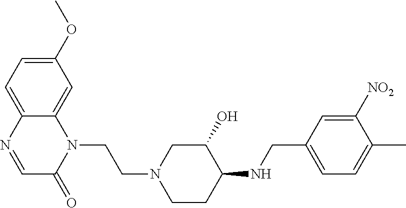 [SMILES: COc1ccc2ncc(=O)n(CCN3CC[C@H](NCc4ccc(C)c([N+](=O)[O-])c4)[C@@H](O)C3)c2c1]

To a stirred solution of 1-(2-((3S,4S)-4-amino-3-hydroxypiperidin-1-yl)ethyl)-7-methoxyquinoxalin-2(1H)-one (0.25 g, 0.760 mmol) and 4-methyl-3-nitrobenzaldehyde (0.128 g, 0.760 mmol) in CHCl₃ (6 ml) was added Sodium Triacetoxyborohydride (0.190 g, 0.930 mmol) at 0° C. The reaction was stirred at ambient temperature for 3 h. Upon completion, the reaction mass was diluted with 10% MeOH—CH₂Cl₂ and washed with water. The organic layers were dried over Na₂SO₄ and concentrated. Silicagel (100-200 mesh) flash column chromatography of the crude eluted the required compound VT-03-00135 with 8% MeOH—CH₂Cl₂ as a pale brown solid (0.17 g).

¹HNMR (400 MHz, DMSO-d₆) δ 8.40 (s, 1H), 8.19 (s, 1H), 7.86 (d, 1H), 7.75 (d, 1H), 7.54 (d, 1H), 7.24-7.34 (m, 2H), 5.51 (s, 1H), 4.55 (t, 2H), 4.20 (t, 2H), 3.90 (s, 3H), 3.61-3.70 (m, 1H), 3.16 (p, 1H), 3.01 (q, 1H), 2.83 (q, 2H), 2.61-2.72 (m, 1H), 2.45 (s, 3H), 2.05 (d, 2H), 2.0 (p, 1H), 1.56 (d, 1H) Mass spectra [M+H]+ m/z 468.30.

### [4] Uses

The compounds of the invention are useful for the treatment of infections in subjects, mammals in particular, including humans. In one embodiment, the compounds may be used for the treatment of infections of soft tissues, blood, skin, mouth, lungs, respiratory tract, urinary tract and reproductive tract.

In another embodiment, the compounds of the invention are useful for the treatment of infections caused by microorganisms, such as but not limited to Staphylococcus species such as Staphylococcus aureus, Staphylococcus epidermidis, Staphylococcus haemolyticus, Enterococcus species such as Enterococcus faecalis, Enterococcus faecium, Streptococcus species like Streptococcus pneumoniae, Streptococcus pyogenes, Streptococcus agalactiae, Moraxella species, for example Moraxella catarrhalis, E. coli, Neisseria meningitidis, Neisseria gonorrhoeae, Klebsiella species such as Klebsiella pneumoniae, Klebsiella oxytoca, Pseudomonas species such as Pseudomonas aeruginosa, Acinetobacter species such as Acinetobacter baumannii.

### [5] Route of Administration

The compounds of the present invention are delivered to the subjects by forms suitable for each administration route. For example, the compounds are administered as tablets, capsules, injection, drops, inhaler, ointment, foams suppository. In a preferred embodiment, the route of administration is oral, parenteral or topical. Topical or transdermal administration include powders, sprays, ointments, pastes creams, lotions, gels, solutions, patches and inhalants.

### [6] Dosage Forms

The composition of the present invention is presented in unit dosage form generally in an amount that produces a therapeutic effect in the subject.

The compounds of the present invention are administered at a daily dose that is the lowest dose effective to produce a therapeutic effect. Generally, the dosage will effect from about 0.0001 to about 100 mg per kg body weight per day. Preferably, the dosage will range from about 0.001 to 75 mg per kg body weight per day and more preferably, the dosage will range from about 0.1 to about 50 mg per kg body weight per day. Each unit dose may be, for example, 5, 10, 25, 50, 100, 125, 150, 200 or 250 mg of the compound of the invention. As per the requirement of the subject, the effective daily dose of the compound is administered as two, three, four or more sub-doses administered separately at appropriate intervals throughout the day, optionally in unit dosage forms.

### [7] Formulation

The antibacterial compositions of the present invention may be administered by any method known in the art. Some examples of suitable modes of administration include oral, intravenous, intramuscular topical or any other parenteral mode of administration.

In certain embodiments, the present invention is directed to a method of formulating compounds of the present invention in a pharmaceutically acceptable carrier or excipient and may be administered in a wide variety of different dosage forms e.g. tablets, capsules, sprays, creams, lotions, ointments, aqueous suspensions syrups, and the like. Such carriers may include one or more of solid diluents or fillers, sterile aqueous media, and various nontoxic organic solvents, etc.

For oral administration, tablets may contain various excipients such as one or more of microcrystalline cellulose, sodium citrate, calcium carbonate and the like, along with various disintegrants such as starch and certain complex silicates, together with granulation binders like polyvinylpyrrolidone, sucrose and the like. Solid compositions of a similar type may also be employed as fillers in gelatin capsules.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluents or solvent e.g. as solution in 1, 3 butane diol. Among the acceptable vehicles and solvents that may be employed are water, Ringer's solution and isotonic sodium chloride solution. In addition, sterile fixed oils are conventionally employed including synthetic mono or diglycerides. In addition, fatty acids such as oleic acid find in the preparation of injectables. These aqueous solutions may be suitable for intravenous injection purposes. The oily solutions may be suitable for intra articular, intramuscular, and/or subcutaneous injection purposes.

In another embodiment, the compounds of the present invention may be administered topically that include transdermal, buccal, or sublingual application. For topical applications, therapeutic compounds may be suitably admixed in a pharmacologically inert topical carrier such as a gel, an ointment, a lotion, and/or a cream. Such topical carriers may include water, glycerol, alcohol, propylene glycol, fatty alcohols, triglycerides, fatty acid esters, and/or mineral oils.

The timing of the administration of the pharmaceutical composition may also be regulated. For example the compounds may be administered intermittently or by controlled release.

### [8] Definitions

As used herein, an “independently” selected substituent refers to a group of substituents, wherein the substituents may be different.

The term “optionally substituted” indicates that the said substituent can be unsubstituted or substituted.

The term “absent” is used to designate the lacking of a group or describe the structural value of a variable. For example in some embodiments, A₂ and A₃ may be null or does not exist. In some other embodiments variable “A₂” for a formula (I) compound, indicates that in the absence of the said variable, the adjacent variables on both sides of the absent variable are connected directly together and is synonymous to a single covalent bond. For example, in the chain-G-A₂-NH-A₃-R₆, if A₂ is “absent”, then the chain becomes-G-NH-A₃-R₆.

The term “alkyl” refers to the radical of saturated aliphatic groups, including straight-chain alkyl groups, branched-chain alkyl groups, cycloalkyl (alicyclic) groups, alkyl substituted cycloalkyl groups and cycloalkyl substituted alkyl groups; wherein the term “cycloalkyl” refers to a saturated or unsaturated monocyclic alkyl ring consisting of 3-8 carbon atoms or a saturated or partially unsaturated bicyclic ring consisting of 9 or 10 carbon atoms.

The term “aryl” refers to a mono- or bicylic aromatic ring containing optionally substituted carbon atoms. The said term“aryl” can be fused to saturated or unsaturated cyclic ring containing minimum one heteroatom selected from oxygen, nitrogen and sulphur which is optionally substituted. The preferred substituents are alkyl, alkoxy, alkyl optionally substituted with alkoxy, alkoxy optionally substituted with alkyl, carboxy, hydroxyalkyl, hydroxyl, halogen, haloalkyl, alkylthio, alkylsulfonyl, cyano, nitro, alkynyl, amino, aminoalkyl, alkylcarbonyl, aminosulfonyl, oxo, carbomyl, carbonyl, haloalkoxy.

The term “heteroaryl” refers to an optionally substituted 5- or 6-membered monocyclic hetero aromatic ring or a 9- or 10-membered bicyclic hetero aromatic ring containing minimum one heteroatom which are independently selected from nitrogen, sulphur or oxygen. The said term“heteroaryl” can be fused to saturated or unsaturated cyclic ring containing minimum one of the said heteroatom which is optionally substituted. The preferred substituents are alkyl, alkoxy, alkyl optionally substituted with alkoxy, alkoxy optionally substituted with alkyl, carboxy, hydroxyalkyl, hydroxyl, halogen, haloalkyl, alkylthio, alkylsulfonyl, cyano, nitro, alkynyl, amino, aminoalkyl, alkylcarbonyl, aminosulfonyl, oxo, carbomyl, carbonyl, haloalkoxy.

The term “alkoxy” refers to alkyl ether radical, wherein the term “alkyl” is as defined above.

The term “amino” refers to —NH₂ group.

The term “aminoalkyl” refers to —NH(alkyl) or —N(alkyl)(alkyl) group wherein the term “alkyl” is as defined above.

The term “aminosulfonyl” refers to —S(═O)₂—NR′₂ radical, wherein each R′ independently represent “alkyl” as defined above or hydrogen.

The term “halogen” refers to F, Cl, Br or I.

The term “haloalkyl” refers to an “alkyl” group substituted with one or more halogen wherein the terms “alkyl” and “halogen” are as defined above.

The term “haloalkoxy” refers to an “alkoxy” group substituted with at least one “halogen” wherein the terms “alkoxy” and “halogen” are as defined above.

The term “hydroxyl” refers to —OH group.

The term “hydroxyalkyl” refers to an alkyl group which is substituted with one or more, preferably one “hydroxyl” group and, wherein the terms “hydroxyl” and “alkyl” are as defined above.

The term “carbomyl” refers to —C(O)NH₂ group.

The term “carbonyl” refers to —C═O group.

The term “oxo” refers to double bonded oxygen atom (═O).

The term “nitro” refers to —NO₂ group.

The term “cyano” refers to —CN group.

The term “carboxy” refers to —C(═O)OH group.

The term “thiol” or “thio” refers to —SH group.

The term “sulfonyl” refers to —S(═O)₂ group.

The term “alkylsulfonyl” refers to —S(═O)₂-alkyl group wherein the term “alkyl” is as defined above.

The term “arylsulfonyl” refers to —S(═O)₂-aryl group wherein the term “aryl” is as defined above.

The term “alkyl sulfonyloxy” refers to —OSO₂-alkyl group wherein the term “alkyl” is as defined above.

The term “aryloxy” refers to aryl ether radical, wherein the term “aryl” is as defined above.

The term “acyloxy” refers to alkyl-C(═O)—O-alkyl where alkyl-C(═O) is the “acyl” group and the term “alkyl” is as defined above.

The term “alkylcarbonyl” refers to —C(═O)(alkyl)- group wherein the term “alkyl” is as defined above.

The term “alkenylcarbonyl” refers to —C(═O)(alkenyl)- group wherein the term “alkenyl” is as defined above.

The term “alkoxycarbonyl” refers to —C(═O)(alkoxy)- group wherein the term “alkoxy” is as defined above.

The term “thioalkyl” or “alkylthio” refers to —S-alkyl radical wherein the term “alkyl” is as defined above.

The term “arylthio” refers to —S-aryl radical wherein the term “aryl” is as defined above.

The term “acylthio” refers to —S-acyl radical wherein the term “acyl” is as defined above.

The term “heterocyclylthio” refers to —S-heterocyclyl radical wherein the term “heterocyclyl” is as defined herein.

The term “heterocyclyloxy” refers to —O-heterocyclyl radical wherein the term “heterocyclyl” is as defined herein.

Unless otherwise defined, the term “heterocyclic” or “heterocyclyl” as used above includes optionally substituted aromatic and non-aromatic, single and fused, mono- or bicyclic rings suitably containing minimum one heteroatom selected from oxygen, nitrogen and sulphur, which rings may be optionally C-substituted. The preferred substituents are alkyl, alkoxy, alkyl optionally substituted with alkoxy, alkoxy optionally substituted with alkyl, carboxy, hydroxyalkyl, hydroxyl, halogen, haloalkyl, alkylthio, alkylsulfonyl, cyano, nitro, alkynyl, amino, aminoalkyl, alkylcarbonyl, aminosulfonyl, oxo, carbomyl, carbonyl, haloalkoxy.

The term “containing at least one heteroatom” refers to at least one carbon atom of the ring being replaced by a heteroatom selected from oxygen, nitrogen and sulphur.

The compounds of present invention may exist in specific geometric or stereoisomeric forms.

The present invention is inclusive of all possible enantiomers and diastereomers in pure or substantially pure form and mixtures of two or more stereoisomers in ratios that are effective. This means that the compounds of present invention may exist both as levorotatory and as dextrorotatory, in the form of racemates and in the form of two enantiomers.

The compounds of present invention are capable of forming both pharmaceutically acceptable salts. Examples of salts include but not restricted to metals or amines such as alkali and alkaline earth metals or organic amines Examples of suitable acids for salt formation include but is not limited to hydrochloric, sulphuric, phosphoric, acetic, citric, oxalic, malonic, salicyclic, malic, fumaric, succinic, ascorbic and the likes thereof.

The compound of the present invention can exist as unsolvated or solvated forms including hydrated forms.

The compounds detailed in the present disclosure are capable of forming pharmaceutically acceptable prodrugs. Prodrugs are covalently bonded carriers that release the active compound internally after administration to the subject.

The present invention provides pharmaceutical compositions comprising an effective amount of compound of Formula (I), prodrugs, tautomeric forms, stereoisomers, optical isomers, pharmaceutically acceptable salts, solvates, polymorphs, analogs or derivatives thereof with pharmaceutically acceptable carriers.

The invention has been described with reference to various specific and preferred embodiments and techniques. However, it should be noted that many variations and modifications may be made while remaining within the scope of the invention.

### Example 1

Analysis of Microbiological Activity of Compounds

Shown below are the microbiological activities of representative compounds of Formula I of the invention. The compounds were tested by the microbroth dilution method (National committee for Clinical Laboratory Standards, 2011, M07-08) and the Minimum Inhibitory Concentration (MIC) was determined.

| | MIC (ug/ml) | | | | | | |
|---|---|---|---|---|---|---|---|
| | | Methicillin | | | | | |
| | | resistant | | | | | |
| | Staphylococcus | Staphylococcus | Streptococcus | Enterococcus | Moraxella | | |
| | aureus | aureus | pneumoniae | faecalis | catarrhalis | E. coli | K. pneumoniae |
| Compound | (ATCC 29213) | (ATCC 33591) | (ATCC 6301) | (ATCC 29212) | (ATCC 8176) | (ATCC 25922) | (ATCC 700603) |
| VT-03- | 2 | 4 | 2 | 4 | 4 | 32 | >128 |
| 00014 | | | | | | | |
| VT-03- | >32 | >32 | >16 | >64 | >16 | >32 | ND |
| 00017 | | | | | | | |
| VT-03- | 0.25 | 0.12 | 1 | 2 | 2 | 16 | >32 |
| 00021 | | | | | | | |
| VT-03- | ≦0.03 | 0.06 | 0.12 | 2 | 2 | 16 | ND |
| 00021a | | | | | | | |
| VT-03- | 1 | 2 | 2 | 4 | 4 | >64 | ND |
| 00022 | | | | | | | |
| VT-03- | 1 | 2 | 2 | 8 | 8 | >32 | ND |
| 00024 | | | | | | | |
| VT-03- | 2 | 4 | 1 | 8 | 2 | 32 | 64 |
| 00026 | | | | | | | |
| VT-03- | 1 | 2 | 2 | 8 | 4 | 16 | ND |
| 00026a | | | | | | | |
| VT-03- | 2 | 4 | 2 | 4 | 8 | >32 | ND |
| 00027 | | | | | | | |
| VT-03- | 0.25 | 0.5 | 0.25 | 1 | 4 | >32 | >32 |
| 00028 | | | | | | | |
| VT-03- | 2 | 4 | 4 | 4 | 1 | 8 | >32 |
| 00030 | | | | | | | |
| VT-03- | 1 | 2 | 1 | 2 | 4 | 16 | >32 |
| 00031 | | | | | | | |
| VT-03- | 2 | 4 | 4 | 8 | 8 | >32 | >32 |
| 00032 | | | | | | | |
| VT-03- | >16 | >16 | ND | ND | ND | >32 | ND |
| 00042 | | | | | | | |
| VT-03- | 16 | >16 | ND | ND | ND | >32 | ND |
| 00043 | | | | | | | |
| VT-03- | 0.06 | 0.12 | 0.5 | 4 | 2 | 32 | ND |
| 00045 | | | | | | | |
| VT-03- | 16 | >16 | ND | ND | ND | >32 | ND |
| 00046 | | | | | | | |
| VT-03- | 0.12 | 0.5 | 0.5 | 4 | 1 | 8 | >32 |
| 00048 | | | | | | | |
| VT-03- | 0.12 | 0.5 | 0.5 | 2 | 8 | 32 | >32 |
| 00049 | | | | | | | |
| VT-03- | 0.25 | 0.5 | 4 | 4 | 4 | 16 | >16 |
| 00050 | | | | | | | |
| VT-03- | 2 | 4 | >4 | 4 | 2 | >32 | >16 |
| 00051 | | | | | | | |
| VT-03- | 0.125 | 0.25 | 1 | 4 | | 16 | |
| 00052 | | | | | | | |
| VT-03- | 0.25 | 0.25 | 1 | 2 | 0.5 | 4 | >32 |
| 00053 | | | | | | | |
| VT-03- | 0.03 | 0.12 | 0.5 | 2 | 1 | 8 | 32 |
| 00054 | | | | | | | |
| VT-03- | 1 | 4 | 0.03 | 0.25 | >32 | >32 | >32 |
| 00055 | | | | | | | |
| VT-03- | 0.25 | 0.5 | 0.25 | 8 | 1 | 4 | >32 |
| 00056 | | | | | | | |
| VT-03- | 0.12 | 0.25 | 0.5 | 2 | 0.5 | 2 | >32 |
| 00057 | | | | | | | |
| VT-03- | 0.015 | 0.06 | 0.03 | 0.25 | 0.5 | 1 | 16 |
| 00058 | | | | | | | |
| VT-03- | 0.25 | 1 | 2 | 8 | 8 | >32 | >32 |
| 00059 | | | | | | | |
| VT-03- | >4 | >4 | ND | ND | >32 | >32 | ND |
| 00060 | | | | | | | |
| VT-03- | 0.015 | 0.015 | 0.015 | 0.25 | 0.12 | 1 | 8 |
| 00061 | | | | | | | |
| VT-03- | ≦0.0075 | 0.015 | 0.015 | 0.12 | ≦0.06 | 0.5 | 8 |
| 00062 | | | | | | | |
| VT-03- | 0.5 | 1 | 0.5 | 4 | 2 | 4 | >32 |
| 00062a | | | | | | | |
| VT-03- | 0.25 | 0.5 | 0.25 | 2 | 0.25 | 4 | >32 |
| 00063 | | | | | | | |
| VT-03- | 0.12 | 0.25 | 0.12 | 2 | 0.25 | 4 | >32 |
| 00064 | | | | | | | |
| VT-03- | 0.25 | 1 | 4 | >32 | >32 | >32 | >32 |
| 00065 | | | | | | | |
| VT-03- | 0.5 | 1 | 1 | 4 | 4 | 16 | >32 |
| 00066 | | | | | | | |
| VT-03- | 0.12 | 0.5 | 0.25 | ND | 2 | 16 | 32 |
| 00067 | | | | | | | |
| VT-03- | >4 | >4 | ND | ND | ND | >4 | ND |
| 00069 | | | | | | | |
| VT-03- | >4 | >4 | ND | ND | ND | >4 | ND |
| 00070 | | | | | | | |
| VT-03- | ND | ND | ND | ND | ND | ND | ND |
| 00071 | | | | | | | |
| VT-03- | >4 | >4 | ND | ND | ND | >4 | ND |
| 00072 | | | | | | | |
| VT-03- | 1 | 2 | 2 | 8 | 2 | 32 | ND |
| 00074 | | | | | | | |
| VT-03- | 0.12 | 0.12 | 0.06 | 0.5 | 0.5 | 4 | >16 |
| 00075 | | | | | | | |
| VT-03- | 0.015 | 0.03 | 0.03 | 0.25 | 0.25 | 2 | >16 |
| 00076 | | | | | | | |
| VT-03- | 0.06 | 0.12 | 0.12 | 1 | 0.25 | 2 | >16 |
| 00077 | | | | | | | |
| VT-03- | >4 | ND | ND | ND | ND | >32 | ND |
| 00078 | | | | | | | |
| VT-03- | 2 | 2 | 2 | 32 | 4 | 32 | ND |
| 00079 | | | | | | | |
| VT-03- | 0.25 | 1 | 2 | 4 | >4 | 32 | ND |
| 00080 | | | | | | | |
| VT-03- | ND | 1 | ND | ND | ND | >32 | ND |
| 00081 | | | | | | | |
| VT-03- | 0.06 | 0.12 | ND | ND | ND | 4 | ND |
| 00083 | | | | | | | |
| VT-03- | 0.5 | 0.5 | 0.25 | 2 | 2 | 8 | ND |
| 00084 | | | | | | | |

ND = not done

### Example 2: MICs Against Fluoroquinolone Resistant Strains

To test if compounds are able to overcome fluoroquinolone resistance, we have determined the MICS against Fluoroquinolone (FQ) resistant clinical strains of MRSA and E. coli.

| Compound name | MIC in μg/ml against FQ resistant strains | |
|---|---|---|
| | MRSA E9823 | MRSA E9749 |
| VT-03-00052 | 0.25 | 0.25 |
| VT-03-00057 | 0.25 | 0.25 |
| VT-03-00061 | ≦0.0075 | 0.015 |
| VT-03-00062 | ≦0.0075 | 0.015 |

| | E. coli | | E. coli | |
|---|---|---|---|---|
| | E1851 | E. coli U1306 | U5690 | E. coli 86 |
| VT-03-00057 | 2 | 16 | 8 | 16 |
| VT-03-00061 | 0.5 | 2 | 2 | 2 |
| Ciprofloxacin | >4 | >4 | >4 | >4 |

### Example 3: Analysis of Target Specificity of Compounds

To test for target specificity, the activity of compounds was evaluated in an in vitro Gyrase assay using recombinant Gyrase protein as per the instructions of the assay kit (Inspiralis). The assay measures the ability of E. coli Gyrase to convert relaxed plasmid DNA into the supercoiled form. The enzyme is incubated with the substrate (relaxed DNA) in the presence and absence of compounds for 1 hour at 37° C. and the DNA is run on a gel at low voltage for several hours. The gel is then stained with Ethidium bromide and DNA in the different forms is quantified using DNA imaging and quantification software (Image Lab). The activity of the enzyme is proportional to the amount of supercoiled form detected.

% inhibition of DNA supercoiling
activity

| Compound | 1 μM | 0.1 μM |
|---|---|---|
| VT-03-00045 | 96.5 | 96.5 |
| VT-03-00048 | 97.3 | 97.5 |
| VT-03-00055 | 71 | 52.7 |
| VT-03-00057 | 90.6 | 61.4 |
| VT-03-00061 | 99.7 | 99.8 |
| VT-03-00062 | 98.7 | 91.5 |
| VT-03-00064 | 98.1 | 89.3 |
| VT-03-00066 | 89.9 | 55.9 |
| VT-03-00077 | 88.5 | 38.8 |
| VT-03-00079 | 87.6 | 16.7 |

### Example 4: Mutation Prevention Concentration Studies

The mutation prevention concentration or the concentration above which mutants are unlikely to be selected, was determined based on published protocols (Antimicrobial Agents and Chemotherapy, 45, 433-438, 2001).

TABLE 2
Mutation prevention concentration for VT-03-00061 against MRSA
33591

| | Mutation prevention concentration |
|---|---|
| Compounds | (μg/ml) |
| VT-03-00061 | 0.12 |

### Example 5: Time Kill Kinetics

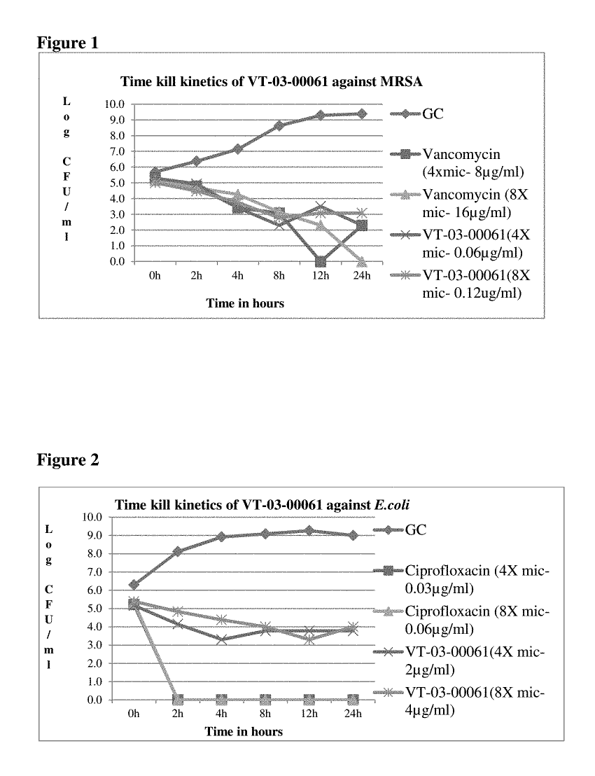

To understand the kinetics of growth in the presence of the VT-03 compounds, we undertook time kill assays (National committee for Clinical Laboratory Standards, M07-A8, Volume 29, 2009). Data for VT-03-00061 against MRSA 33591 (FIG. 1: time kill kinetics of VT-03-00061 against MRSA) and E. coli 25922 (FIG. 2: time kill kinetics of VT-03-00061 against E. coli); GC=Growth control.

### Example 6: hERG Binding Studies

Inhibition of the inward rectifying voltage gated potassium channel encoded by the human ether-a-go-go related gene (hERG) current causes QT interval prolongation which may lead to cardiac arrhythmia (Current Topics in Ion Channels, 2008, 2, 87-93). To test the ability of the VT-03 compounds to bind the hERG channel, membranes expressing the hERG channel were incubated with radiolabeled Astemizole and displacement of the labeled ligand in the presence of compounds was measured. These data were used to derive the concentration at which 50% of the radioligand is displaced (IC₅₀). The compounds show no significant hERG binding activity up to the highest concentration tested indicating the advantage of VT-03 compounds over the known prior art antibacterial compounds (ref: BMCL, 21, 7489-7495, 2011).

| | | Highest test |
|---|---|---|
| Compound Name | IC₅₀ value | concentration (μM) |
| VT-03-00063 | >30.00 μM | 30 |
| VT-03-00058 | >30.00 μM | 30 |
| VT-03-00061 | >30.00 μM | 30 |
| VT-03-00053 | >30.00 μM | 30 |

### Example 7: Pharmacokinetic Profiles

Compounds were dosed to male Swiss albino mice to determine the pharmacokinetic profiles. Data for VT-03-00061 are shown below. The compound is orally bioavailable.

TABLE 5
Pharmacokinetic profiles of select compounds
Single Dose Pharmacokinetics Study of VT-03-00061 in
Male Swiss Albino Mice

| | Oral PK Study | Intravenous PK Study |
|---|---|---|
| PK Parameters | (10 mg/kg b.w.) | (5 mg/kg b.w.) |
| Cₘₐₓ (ng/mL) | 51.47 | 400.18 |
| AUC_{inf} (h * ng/mL) | 103.82 | 368.41 |
| T_{1/2} (h) | 1.74 | 1.64 |

| | |
|---|---|
| F % (Oral bioavaialbility) | 11.51 |

### Example 8: In Vivo Activity in the Systemic Infection Model Against S. aureus (MRSA ATCC 33591)

In order to evaluate the in vivo efficacy of the scaffold, we tested representative compounds for activity in the systemic infection model in mice (Antimicrobial Agents and Chemotherapy, 47, 2507-2512, 2003). In this model, a 15× medial lethal dose of the bacteria (MRSA ATCC33591) is administered to mice intraperiotoneally. An hour later, the compound is administered i.v. and again 4 hours later. VT-03-00061, was efficacious with a 50% survival at a dose of 10 mg/kg in this model.

### Example 9

Analysis of microbiological activity of compounds shown below are the microbiological activities of representative compounds of Formula II of the invention. The compounds were tested by the microbroth dilution method as per the reference in Example 1.

To test for target specificity, the activity of compounds was evaluated in an in vitro Gyrase supercoiling assay using ciprofloxacin resistant recombinant Gyrase protein (with mutation S83L) as per the instructions of the assay kit (Inspiralis). The enzyme is incubated with the substrate (relaxed DNA) in the presence and absence of compounds for 1 hour at 37° C. and the DNA is run on a gel at low voltage for several hours. The amount of supercoiled DNA is quantified using DNA imaging and quantification software (Image Lab). The activity of the enzyme is proportional to the amount of supercoiled form detected.

| | Antibacterial activity | | | | | | | |
|---|---|---|---|---|---|---|---|---|
| | Minimum Inhibitory Concentration (MIC) (μg/ml) | | | | | | Target specificity: | |
| | | | | Klebsiella | | | % inhibition of | |
| | MRSA | E. faecalis | E. coli | KP | Acinetobacter | Pseudomonas | supercoiling activity | |
| Compound | ATCC 33591 | ATCC 51299 | ATCC 25922 | 700603 | BAA747 | ATCC 27853 | 1 μM | 0.1 μM |
| VT-03- | 4 | 16 | >16 | >16 | NT | NT | 92.3 | 95.9 |
| 00101 | | | | | | | | |
| VT-03- | ≦0.0075 | 0.5 | 8 | >16 | NT | NT | 95.3 | 96.5 |
| 00102 | | | | | | | | |
| VT-03- | 0.03 | 0.25 | 0.5 | 8 | 1 | 32 | 87.48 | 55.8 |
| 00103 | | | | | | | | |
| VT-03- | 0.06 | 0.5 | 1 | 8 | NT | NT | NT | NT |
| 00107 | | | | | | | | |
| VT-03- | 0.25 | 2 | 8 | >32 | NT | NT | NT | NT |
| 00108 | | | | | | | | |
| VT-03- | 1 | 2 | 16 | >32 | NT | NT | NT | NT |
| 00109 | | | | | | | | |
| VT-03- | 0.125 | 0.5 | 1 | >16 | NT | NT | 87.64 | 90.42 |
| 00111 | | | | | | | | |
| VT-03- | 0.125 | NT | 2 | 32 | NT | NT | NT | NT |
| 00112 | | | | | | | | |
| VT-03- | 0.03 | 0.25 | 0.125 | 8 | 0.25 | 8 | 93.97 | 89.95 |
| 00113 | | | | | | | | |
| VT-03- | 0.06 | 0.5 | 2 | 32 | NT | NT | NT | NT |
| 00114 | | | | | | | | |
| VT-03- | 0.03 | 0.25 | 0.25 | 8 | 0.25 | 16 | 94.28 | 96.91 |
| 00116 | | | | | | | | |
| VT-03- | 0.125 | 0.25 | 0.5 | 8 | 0.25 | 16 | 97.55 | 88.2 |
| 00117 | | | | | | | | |
| VT-03- | 0.015 | 0.125 | 0.25 | 8 | 0.25 | 8 | 88.97 | 91.46 |
| 00118 | | | | | | | | |
| VT-03- | 4 | 16 | 8 | >32 | 8 | >32 | NT | NT |
| 00119 | | | | | | | | |
| VT-03- | 0.06 | 0.5 | 0.25 | 8 | 0.25 | 16 | 97.08 | 95.45 |
| 00122 | | | | | | | | |
| VT-03- | NT | NT | 2 | 32 | NT | NT | NT | NT |
| 00124 | | | | | | | | |
| VT-03- | >4 | NT | 32 | NT | NT | NT | No | No |
| 00125 | | | | | | | inhibition | inhibition |
| VT-03- | 0.5 | NT | 2 | 32 | 8 | >32 | NT | NT |
| 00126 | | | | | | | | |
| VT-03- | 0.125 | 1 | 1 | 8 | 1 | 8 | 72% | 39% |
| 00127 | | | | | | | | |
| VT-03- | 0.015 | 0.25 | 0.5-1 | 16 | 0.25 | 8 | 51% | 34% |
| 00128 | | | | | | | | |
| VT-03- | NT | NT | 4 | >32 | 4 | >32 | NT | NT |
| 00129 | | | | | | | | |
| VT-03- | 0.25 | 0.25 | 0.25-1 | 8 | 0.25 | 16 | NT | NT |
| 00131 | | | | | | | | |
| VT-03- | 1 | 1 | 2 | NT | 2 | 32 | NT | NT |
| 00132 | | | | | | | | |
| VT-03- | 0.015 | 0.25 | 2 | 32 | 0.5 | 32 | NT | NT |
| 00134 | | | | | | | | |
| VT-03- | NT | NT | 8 | NT | NT | NT | NT | NT |
| 00135 | | | | | | | | |

### Example 10

### Mutation Prevention Concentration Studies

The mutation prevention concentration and mutation frequencies were determined based on published protocols, the reference of which is provided in Example 4.

| | Mutation | |
|---|---|---|
| | prevention concentration | Mutation |
| Compounds | (μg/ml) against A. baumannii | frequency |
| VT-03-00122 | 16 | 1.10 * 10⁻⁸ at 8 μg/ml |

### Example 11

hERG Binding Studies

To test the ability of the VT-03 compounds (of Formula II) to bind the hERG channel, membranes expressing the hERG channel were incubated with radiolabeled Astemizole and displacement of the labeled ligand in the presence of compounds was measured. The table below shows IC₅₀ concentrations. The compounds show no significant hERG binding activity up to the highest concentration tested.

| Compound Name | IC₅₀ value | Highest test concentration (μM) |
|---|---|---|
| VT-03-00118 | >30 μM | 30 μM |
| VT-03-00122 | >30 μM | 30 μM |

### Example 12

### Pharmacokinetic Profiles

Compounds were dosed to male Swiss albino mice to determine the pharmacokinetic profiles. Results suggest that the compounds are orally bioavailable

| | VT-03-00113 | | VT-03-00118 | | VT-03-00122 | | VT-03-00127 | |
|---|---|---|---|---|---|---|---|---|
| | oral | i.v. | oral | i.v. | oral | i.v. | oral | i.v. |
| Dose (mg/kg) | 10 | 5 | 10 | 5 | 10 | 5 | 10 | 5 |
| Cmax (ng/ml) | 15.55 | 672.29 | 18.62 | 912.98 | 28.06 | 381.44 | 96.99 | 478.43 |
| Tmax (hr) | 0.50 | 0.16 | 1 | 0.16 | 1 | 0.16 | 0.50 | 0.16 |
| AUCₗₐₛₜ(hr*ng/mL) | 9.3 | 401.94 | 9.3 | 496.79 | 37.33 | 241.66 | 198.36 | 526.99 |
| T_{1/2} (hour) | NC | 0.37 | 11.10 | 0.37 | NC | 0.49 | 2.89 | 2 |

| Oral | 1.16 | 1.12 | 7.69 | 18.82 |
|---|---|---|---|---|
| bioavailability | | | | |

We claim:

1. A compound of Formula (II) or a pharmaceutically acceptable salt thereof, 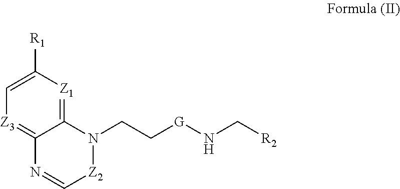
wherein Z₁ and Z₃ are each independently selected from a group consisting of CH or N;
Z₂ is independently selected from group consisting of C═O and C═S;
R₁ is independently selected from the group consisting of hydrogen, methoxy, cyano, halogen, hydroxyl, C₁₋₆ alkoxy and C₁₋₆ alkyl optionally substituted with one or two C₁₋₆ alkoxy, alkyne, carboxyl, carboxamide;
G is G1, G2 or G3; 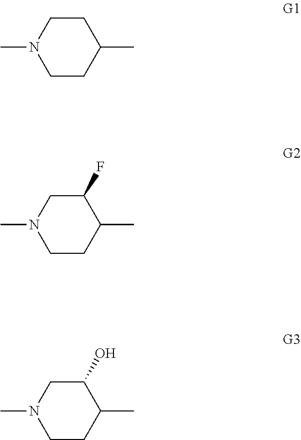
and wherein, R₂ is selected from the group consisting of
(i) a substituted or unsubstituted monocylic aryl; and
(ii) a substituted or unsubstituted monocylic heteroaryl.

2. A compound selected from the group consisting of:
VT-03-000100:1-(2-(4-(3-fluoro-4-methylbenzylamino)piperidin-1-yl)ethyl)-7-methoxy quinoxalin-2(1H)-one;
VT-03-000101: 4-(2-(4-(3-fluoro-4-methylbenzylamino)piperidin-1-yl)ethyl)-6-methoxy pyrido[3,2-b]pyrazin-3(4H)-one;
VT-03-000102:7-bromo-1-(2-(4-(3-fluoro-4-methylbenzylamino)piperidin-1-yl)ethyl)-quinoxalin-2(1H)-one;
VT-03-000103:4-(2-(4-(3-fluoro-4-methylbenzylamino)piperidin-1-yl)ethyl)-3-oxo-3,4-dihydroquinoxaline-6-carbonitrile;
VT-03-000104:4-(2-(4-(4-methyl-3-nitrobenzylamino)piperidin-1-yl)ethyl)-3-oxo-3,4-dihydroquinoxaline-6-carbonitrile;
VT-03-000106:6-methoxy-4-(2-(4-(4-methyl-3-nitrobenzylamino)piperidin-1-yl)ethyl)pyrido-[3,2-b]pyrazin-3(4H)-one;
VT-03-000107:6-chloro-4-(2-(4-(3-fluoro-4-methylbenzylamino)piperidin-1-yl)ethyl)-1,2-dihydropyrido[3,2-b]pyrazin-3(4H)-one
VT-03-000108: N-methyl-4-(2-(4-(4-methyl-3-nitrobenzylamino)piperidin-1-yl)ethyl)-3-oxo-3,4-dihydroquinoxaline-6-carboxamide;
VT-03-000109:4-(2-(4-(3-fluoro-4-methylbenzylamino)piperidin-1-yl)ethyl)-N-methyl-3-oxo-3,4-dihydroquinoxaline-6-carboxamide;
VT-03-000110:7-ethynyl-1-(2-(4-(3-fluoro-4-methylbenzylamino)piperidin-1-yl)ethyl)-quinoxalin-2(1H)-one;
VT-03-000111:6-chloro-4-(2-(4-(3-fluoro-4-methylbenzylamino)piperidin-1-yl)ethyl)pyrido-[3,2-b]pyrazin-3(4H)-one;
VT-03-000112:7-ethyl-1-(2-(4-(3-fluoro-4-methylbenzylamino)piperidin-1-yl)ethyl)quinoxalin-2(1H)-one;
VT-03-000113:7-fluoro-1-(2-(4-(4-methyl-3-nitrobenzylamino)piperidin-1-yl)ethyl)quinoxalin-2(1H)-one;
VT-03-000114:1-(2-(4-(4-methyl-3-nitrobenzylamino)piperidin-1-yl)ethyl)-7-(trifluoromethoxy-quinoxalin-2(1H)-one;
VT-03-000115:1-(2-(4-(4-methyl-3-nitrobenzylamino)piperidin-1-yl)ethyl)quinoxalin-2(1H)one;
VT-03-000116:1-(2-(4-(4-chloro-3-nitrobenzylamino)piperidin-1-yl)ethyl)-7-fluoroquinoxalin-2(1H)-one;
VT-03-000117:5-((1-(2-(7-fluoro-2-oxoquinoxalin-1(2H)-yl)ethyl)piperidin-4-ylamino) methyl)-2-methylbenzonitrile;
VT-03-000118:6-chloro-4-(2-(4-(4-methyl-3-nitrobenzylamino)piperidin-1-yl)ethyl)pyrido[3,2-b]pyrazin-3(4H)-one;
VT-03-000119:1-(2-(4-(4-methyl-3-nitrobenzylamino)piperidin-1-yl)ethyl)-2-oxo-1,2-dihydropyrido[2,3-b]pyrazine-7-carbonitrile;
VT-03-000120:1-(2-(4-(4-methyl-3-nitrobenzylamino)piperidin-1-yl)ethyl)pyrido[2,3-b]pyrazin-2(1H)-one;
VT-03-000121:6-fluoro-4-(2-(4-(4-methyl-3-nitrobenzylamino)piperidin-1-yl)ethyl)pyrido[3,2-b]pyrazin-3(4H)-one;
VT-03-000122:7-fluoro-1-(2-(4-(3-fluoro-4-methylbenzylamino)piperidin-1-yl)ethyl)quinoxalin-2(1H)-one;
VT-03-000123:4-(2-(4-(4-methyl-3-nitrobenzylamino)piperidin-1-yl)ethyl)-3-oxo-3,4-dihydroquinoxaline-6-carboxylic acid;
VT-03-000124:4-(2-(4-(4-methyl-3-nitrobenzylamino)piperidin-1-yl)ethyl)-3-oxo-3,4-dihydroquinoxaline-6-carboxylic acid;
VT-03-000125:6-methoxy-4-(2-(4-(4-methyl-3-nitrobenzylamino)piperidin-1-yl)ethyl)-pyrido[3,2-b]pyrazin-3(4H)-one;
VT-03-000126:1-(2-(4-(3-fluoro-4-methylbenzylamino)piperidin-1-yl)ethyl)-7-methoxyquinoxaline-2(1H)-thione;
VT-03-000127:7-methoxy-1-(2-(4-(4-methyl-3-nitrobenzylamino)piperidin-1-yl)ethyl)quinoxaline-2(1H)-thione;
VT-03-000128:1-(2-(4-(4-chloro-3-nitrobenzylamino)piperidin-1-yl)ethyl)-7-methoxyquinoxalin-2(1H)-on;
VT-03-000131:5-((1-(2-(6-chloro-3-oxopyrido[3,2-b]pyrazin-4(3H)-yl)ethyl)piperidin-4-ylamino)-methyl)-2-methylbenzonitrile;
VT-03-000132:5-((1-(2-(7-methoxy-2-thioxoquinoxalin-1(2H)-yl)ethyl)piperidin-4-ylamino)-methyl)-2-methylbenzonitrile;
VT-03-000133:5-((1-(2-(7-methoxy-2-oxoquinoxalin-1(2H)-yl)ethyl)piperidin-4-ylamino)-methyl)-2-methylbenzonitrile;
VT-03-000134:1-(2-((3R,4S)-3-fluoro-4-(4-methyl-3-nitrobenzylamino)piperidin-1-yl)ethyl)-7-methoxyquinoxalin-2(1H)-one; and
VT-03-000135:1-(2-((3S,4S)-3-hydroxy-4-(4-methyl-3-nitrobenzylamino)piperidin-1-yl)ethyl)-7-methoxyquinoxalin-2(1H)-one.

3. A method of treating a patient suffering from an infection comprising administering an effective amount of a compound of claim 1, wherein the infection is caused by at least one of Staphylococcus species, Enterococcus species, Streptococcus species, Moraxella species, E. coli, Neisseria meningitidis, Neisseria gonorrhoeae, Klebsiella species, Pseudomonas species, or Acinetobacter species.

4. A method of treating a patient suffering from an infection comprising administering an effective amount of a compound of claim 2, wherein the infection is caused by at least one of Staphylococcus species, Enterococcus species, Streptococcus species, Moraxella species, E. coli, Neisseria meningitidis, Neisseria gonorrhoeae, Klebsiella species, Pseudomonas species, or Acinetobacter species.
